(19) United States
(12) Patent Application Publication
(10) Pub. No.: US 2011/0262365 A1
(43) Pub. Date: Oct. 27, 2011

(54) METHODS OF DIAGNOSING HYPERSENSITIVITY TO A FEMALE REPRODUCTIVE HORMONE AND TREATING MEDICAL CONDITIONS ASSOCIATED WITH SAME

(76) Inventors: Alek Itsekson, Yahud (IL); Matetyahu Zolti, Ganei-Tikva (IL); Igor Derzy, Petach-Tikva (IL)

(21) Appl. No.: 13/128,890

(22) PCT Filed: Nov. 12, 2009
(86) PCT No.: PCT/IL09/01069

§ 371 Date: Jul. 18, 2011

Related U.S. Application Data
(60) Provisional application No. 61/193,289, filed on Nov. 13, 2008.

(51) Int. Cl. A61K 38/22; A61K 31/57; A61P 15/06; A61P 15/08; A61P 25/06; A61K 49/00; A61P 15/00

(52) U.S. Cl. 424/9.8; 424/9.1; 514/9.8; 514/177

(57) ABSTRACT

Provided are methods of diagnosing a medical condition associated with hypersensitivity to a female reproductive hormone in a subject, with the proviso that the medical condition is not estrogen dermatitis. The method is effected by administering into a skin of the subject at least one female reproductive hormone such as estriol or estrone, and monitoring a reaction to the female reproductive hormone on the skin, wherein a presence of the reaction above a predetermined threshold is indicative of the presence of the medical condition in the subject. Also provided are methods and computing platforms for treating the medical conditions associated with the hypersensitivity to the female reproductive hormone by desensitization.

### FIELD AND BACKGROUND OF THE INVENTION

The present invention, in some embodiments thereof, relates to methods of diagnosing hypersensitivity to a female reproductive hormone and treating medical conditions associated with hypersensitivity to the female reproductive hormone.

Premenstrual syndrome (PMS) is characterized by significant mood, behavioral and physical changes that occur several days to two weeks before menses (i.e., within the luteal phase of the menstrual cycle) and abate during the menstrual flow. Although most menstruating women experience signs and symptoms linked to the menstrual cycle, the majority of these signs and symptoms are mild and reflect normal physiologic changes. Women with clinically significant PMS usually experience more than one symptom, and on average those seeking treatment report 3 or more troublesome symptoms. The most frequently reported symptoms include mood symptoms (e.g., irritability, anxiety, nervous tension, mood swings, feeling out of control, and a depressed mood) and decreased interest in usual activities, fatigue, poor concentration, poor sleep, and physical changes such as breast tenderness and abdominal swelling. The current estimates of the prevalence of clinically significant PMS (moderate to severe PMS) vary from 12.6% to 31% of menstruating women. The morbidity of PMS is due to severity of symptoms, chronicity (long duration) and the resulting emotional distress or impairment in relationships, activities and work (e.g., 10-15% of PMS patient are unable to work). Current clinical evidence suggests that PMS tends to be a chronic illness with little spontaneous recovery.

A more serious clinical condition is premenstrual dysphoric disorder (PMDD), which affects 5% to 8% of menstruating women. The diagnosis of PMDD is based on a patient reporting at least 5 specific symptoms listed in the DSM-IV criteria. Criteria include physical symptoms, such as breast tenderness, joint pain and mood symptoms, such as marked anxiety, persistent irritability, or feelings of hopelessness. Data have indicated that an individual diagnosed with PMDD experiences 3.8 years of disability during her reproductive years.

In the past, PMS treatment included dietary changes, such as discontinuance of all caffeine-containing products, low carbohydrate diet, calcium supplements, Vitamin B6 (pyridoxine) and progesterone. However, such treatments had no significant effect. In addition, various drugs are currently used for relieving PMS symptoms. These include antidepressant drugs such as fluoxetine (PROZAC), sertraline (ZOLOFT), paroxetine (PAXIL) and clomipramine (ANAFRANIL); anti anxiety drugs such as alprazolam (XANAX) and buspirone (BUSPAR); and ovulation suppression drugs such as the GnRH agonists LUPRON BUSERELIN. However, in many cases symptoms that improve during medical treatment worsen quickly after treatment is stopped.

The underlying causes of PMS/PMDD are thought to result from both hormonal and serotonergic etiologies, although the exact cause of PMDD has not been fully elucidated.

Schmidt et al. (1998), demonstrated that the occurrence of PMS symptoms represents an abnormal response to normal hormonal changes. In addition, skin tests for abnormal sensitivity to estrogen and progesterone were described in patients having pruritus vulvae (Govorukhina E M, 1986) or premenstrual syndrome (Govorukhina E M, 1987; Itsekson A et al., 2004).

Desensitization with estrogen or progesterone was suggested for treating pre-menstrual asthma and menstrual migraines (Roby et al., 2006), pruritus vulvae, premenstrual syndrome (Miller J B, 1974; Mabray C R et al., 1982) and infertility (U.S. Patent Appl. 20050065136 to Roby Russell R).

Additional background art includes Wilkinson S M and Beck M H, 1994; Beaumont V, et al., 1992; Lindsay S. 2006; Davis D L, et al., 1997; Schoenmakers A., et al., 1992, 26:159-62; and Battist A P., and Baldwin J L., 2004; Stranahan D., et al., 2006; Shelley W B., et al., 1995; Roby R. Russell U.S. Patent Appl. 20060287285; Itsekson A., et al. 2007; US Patent Application No. 20040067244.

### SUMMARY OF THE INVENTION

According to an aspect of some embodiments of the present invention there is provided a method of diagnosing a medical condition associated with hypersensitivity to a female reproductive hormone in a subject, with the proviso that the medical condition is not estrogen dermatitis, comprising: (a) administering into a skin of the subject at least one female reproductive hormone, wherein the at least one female reproductive hormone comprises estriol or estrone, and; (b) monitoring a reaction to the female reproductive hormone on the skin, wherein a presence of the reaction above a predetermined threshold is indicative of the presence of the medical condition in the subject.

According to an aspect of some embodiments of the present invention there is provided a kit for diagnosing a medical condition associated with hypersensitivity to a female reproductive hormone in a subject, the kit comprising at least one female reproductive hormone, wherein the at least one female reproductive hormone comprises estriol or estrone, and a lesion indicator for assessing a reaction to the female reproductive hormone on a skin.

According to an aspect of some embodiments of the present invention there is provided a method of treating a medical condition associated with hypersensitivity to a female reproductive hormone in a subject, comprising: (a) diagnosing the medical condition according to the method of the invention; and (b) subjecting the subject to a desensitization treatment to the hypersensitivity to the female reproductive hormone.

According to an aspect of some embodiments of the present invention there is provided a computing platform for designing a treatment regimen against hypersensitivity to a female reproductive hormone, comprising: (a) a data storage device storing data comprising skin test results of a subject against the female reproductive hormone (b) a processing unit for designing treatment regimen against the hypersensitivity to the female reproductive hormone using the data stored on the data storage devise; (c) a user interface being for displaying the treatment regimen.

According to some embodiments of the invention, the at least one female reproductive hormone further comprises estradiol and/or progesterone.

According to some embodiments of the invention, the lesion indicator determines presence of the reaction above a predetermined threshold.

According to some embodiments of the invention, the predetermined threshold comprises an increase in diameter of at least 5 mm in the reaction as compared to a control test.

According to some embodiments of the invention, the medical condition comprises a premenstrual syndrome.

According to some embodiments of the invention, the medical condition is selected from the group consisting of premenstrual dysphoric disorder (PMDD), pruritus vulvae, menstrual asthma, menstrual migraine, infertility, hot flashes, mastalgia, acne, premenopausal syndrome, menopausal syndrome, recurrent abortions, fatigue syndrome, unexplained preterm delivery, unexplained irritable bowel syndrome, endometriosis, idiopathic premature delivery, sympathovagal dysbalance in pregnancy (hypotension and/or hypertension), menstrual cycle related alterations in metabolic diseases (e.g. Diabetes), hyper-emesis gravidarum, idiopathic gingivitis, unexplained infertility, unexplained recurrent in vitro fertilization (IVF) failure, unexplained recurrent intrauterine insemination (IUI) failure, unexplained IVF side effects, unexplained IUI side effects, unexplained ovarian hyperstimulation, unexplained male infertility, hypomagnesaemia, menstrual related catamenial epilepsy, menstrual related pain condition, unexplained pain condition, fibromyalgia dysmenorrhea, migraine, menstrual cycle associated hyperosmia, menstrual cycle associated accidents, menstrual cycle associated confusion, menstrual cycle associated weight gain, premature ovarian failure, and unexplained ovarian failure.

According to some embodiments of the invention, the data further comprises anamnesis data of the subject.

According to some embodiments of the invention, the anamnesis data is selected from the group consisting of body mass index (BMI), birth weight, regularity of menstrual cycle and gestational age at birth.

According to some embodiments of the invention, the data further comprises subject's general sense of well being.

According to some embodiments of the invention, the treatment regimen comprises desensitization treatment.

According to some embodiments of the invention, the desensitization treatment comprises three consecutive administrations of the female reproductive hormone into the skin of the subject.

According to some embodiments of the invention, the three consecutive administrations include a first administration at a dosage of 0.1-55 μg of the female reproductive hormone, a second administration at an identical or larger dosage of the first administration and a third administration at an identical or larger dosage of the second administration.

According to some embodiments of the invention, the subject has a menstrual cycle with regular ovulation, and whereas an interval between the three consecutive administrations comprises a complete menstrual cycle.

According to some embodiments of the invention, the subject is at menopause, and whereas an interval between the three consecutive administrations comprises one week.

According to some embodiments of the invention, an interval between the three consecutive administrations comprises one week.

According to some embodiments of the invention, the subject has a menstrual cycle with regular ovulation and whereas the first administration of the three consecutive administrations is effected during a luteal phase of the menstrual cycle, the second administration of the three consecutive administrations is effected following one week of the first administration, and the third administration of the three consecutive administrations is effected following one week of the second administration.

According to some embodiments of the invention, the subject is a non-fertile female.

According to some embodiments of the invention, the subject has an irregular menstrual cycle or an unovulation.

According to some embodiments of the invention, the subject has a non-menstrual cycle related medical condition.

According to some embodiments of the invention, the subject has a menstrual cycle with regular ovulation, and whereas the first administration of the three consecutive administrations is effected during a luteal phase of the menstrual cycle, the second administration of the three consecutive administrations is effected following a complete menstrual cycle of the first administration, and the third administration of the three consecutive administrations is effected to the subject following a complete menstrual cycle of the second administration.

According to some embodiments of the invention, the female reproductive hormone is selected from the group consisting of estradiol, estrone, estriol and progesterone.

According to some embodiments of the invention, the female reproductive hormone is selected from the group consisting of estradiol, estrone and progesterone, and whereas the administration includes a dose of 0.1-25 μg of the female reproductive hormone.

According to some embodiments of the invention, the female reproductive hormone is estriol, and whereas the administration includes a dose of 0.1-55 μg of the female reproductive hormone.

According to some embodiments of the invention, the female reproductive hormone is estriol, and whereas said administration includes a dose of about 0.1-55 μg of said female reproductive hormone.

According to some embodiments of the invention, the administration is effected by an intradermal injection.

According to some embodiments of the invention, the administration is effected by a patch reservoir.

According to some embodiments of the invention, the administration is effected by a matrix reservoir.

According to some embodiments of the invention, the administration is effected using a gel.

According to some embodiments of the invention, the administration is effected using a liquid spray.

According to some embodiments of the invention, the administration is effected on a breached skin using a patch reservoir.

According to some embodiments of the invention, the administration is effected on an intact skin using a patch reservoir.

According to some embodiments of the invention, the user interface being further for recording symptoms of the medical condition.

According to some embodiments of the invention, the user interface being further for recording subject's general sense of well being.

According to some embodiments of the invention, the user interface being further for recording reaction on the skin to the female reproductive hormone.

According to some embodiments of the invention, the control test comprises administering into the skin a diluent of the female reproductive hormone.

According to some embodiments of the invention, the diluent comprises ethyl oleate.

According to some embodiments of the invention, the diluent is a biocompatible diluent.

According to some embodiments of the invention, the diluent comprises a synthetic oil, a vegetal oil, an animal oil or combination thereof.

According to some embodiments of the invention, the diluent comprises a vegetable or a fruit oil.

According to some embodiments of the invention, the diluent further comprises alcohol.

According to some embodiments of the invention, the diluent further comprises an enhancer for increasing permeablization of the skin.

According to some embodiments of the invention, the diluent further comprises benzyl alcohol or ethyl alcohol.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic flow chart depicting an algorithm for diagnosing and optionally treating a medical condition associated with hypersensitivity to a female reproductive hormone according to some embodiments of the invention. Subject selection (210) for a skin test is based on the anamnesis data of the subject (which include clinical symptoms and medical history). Exclusion criteria include symptoms of estrogen dermatitis and or prior diagnosis of estrogen dermatitis. Inclusion criteria include symptoms of a medical condition such as premenstrual syndrome (PMS), premenstrual dysphoric disorder (PMDD), pruritus vulvae, menstrual asthma, menstrual migraine, infertility, hot flashes, mastalgia, acne, premenopausal syndrome, menopausal syndrome (hot flashes and night sweats), recurrent abortions, fatigue syndrome, unexplained preterm delivery, unexplained irritable bowel syndrome, endometriosis, idiopathic premature delivery, sympathovagal dysbalance in pregnancy (hypo- and hypertension), menstrual cycle related alterations in metabolic diseases (e.g. Diabetes), hyper emesis gravid arum, idiopathic gingivitis, unexplained infertility, unexplained recurrent in vitro fertilization (IVF) failure, unexplained recurrent intrauterine insemination (IUI) failure, unexplained IVF side effects, unexplained IUI side effects, unexplained ovarian hyperstimulation, unexplained male infertility, hypomagnesemia, menstrual related catamenial epilepsy, menstrual related pain condition, unexplained pain condition, fibromyalgia, dysmenorrhea, migraine, menstrual cycle associated hyperosmia, menstrual cycle associated accidents, menstrual cycle associated confusion, menstrual cycle associated weight gain, premature ovarian failure, and unexplained ovarian failure. The skin test (220) determines presence or absence of a hypersensitivity to estriol and optionally to other female reproductive hormones such as estradiol, estrone and/or progesterone. The results of the skin test along with the anamnesis data enable the diagnosis of the medical condition of the subject. Based on the diagnosis, the subject can be treated (230) by desensitization of the hypersensitivity to the female reproductive hormone or by other method known in the art.

FIG. 2 is a schematic illustration of computing platform 100 according to some embodiments of the invention. Computing platform 100 includes data storage device 110 for storing data comprising clinical data of the subject including skin test results, processing unit 120 for diagnosing hypersensitivity to a female reproductive hormone and/or designing treatment regimen using the data stored on data storage devise 110; and user interface 140 for displaying patient's diagnosis and/or the treatment regimen. Data storage device 110, processing unit 120 and user interface 140 are connected preferably in a two-way mode. User interface 140 is connected to recorder 150, which records the reaction on the skin to the female reproductive hormone and/or the symptoms of the medical condition.

FIG. 3 is a schematic flow chart depicting an algorithm for executing treatment according to some embodiments of the invention. Clinical symptom(s) data is collected (1, 2) along with anamnesis data (3, 2), the data is used to screen for patients' qualification for further testing and treatment (4). Patients which do not qualify to the skin test [i.e., (NO)] are forwarded to other treatment modalities (5). Patients who undergo the skin test are monitored and data comprising reaction data (7) and clinical symptoms (8) is collected (6). Data of 6, 7 and 8 is processed (9) to produce the dosages and timing of hormone desensitization treatment, and selection of type of hormone for desensitization (10). During the desensitization additional data is collected (11) resulting from surveillance after the skin reaction (12) and Visual Analogue Score (VAS) scores of clinical symptoms (13). The VAS and skin reaction are scored to determine alleviation of symptoms (14). Sufficient improvement will result in termination of treatment (15). Insufficient improvement after fifth treatment (16) cycle is considered unsuccessful treatment (17). Insufficient improvement prior to the fifth treatment cycle is followed by re-evaluation of VAS scores and skin reaction (18) and if necessary changes in the treatment regimen for additional treatment cycle (19). Upon completion of this new cycle steps 11 and on may be repeated.

### DESCRIPTION OF SPECIFIC EMBODIMENTS OF THE INVENTION

The present invention, in some embodiments thereof, relates to methods of diagnosing and treating a medical condition associated with hypersensitivity to a female reproductive hormone and to a computing platform for designing a treatment regimen against the hypersensitivity to the female reproductive hormone.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have designed a novel method for the detection of a hypersensitivity to a female reproductive hormone which can be used to diagnose various medical conditions such as premenstrual syndrome, pruritus vulvae, infertility and various unexplained medical conditions such as unexplained preterm delivery, irritable bowel syndrome and menstrual-related pain conditions (mastalgia, dysmenorrhea and migraine).

As shown in Example 1 of the Examples section which follows, the novel method developed by the present inventors is based on administering into a skin hormones such as estriol, estrone, estradiol (e.g., 17 beta estradiol valerate) and/or progesterone and monitoring the immediate (e.g., during and after 20 minutes), late (during and after 24 hours) and delayed (e.g., during and after 48 hours, a week/month, cell-mediated immunological reaction) type hypersensitivity responses to the injected hormone.

According to an aspect of some embodiments of the invention there is provided a method of diagnosing a medical condition associated with a hypersensitivity of a subject to a female reproductive hormone. The method is effected by (a) administering into a skin of the subject at least one female reproductive hormone, wherein the at least one female reproductive hormone comprises estriol or estrone, and; (b) monitoring a reaction to the female reproductive hormone on the skin, wherein a presence of the reaction above a predetermined threshold is indicative of the presence of the medical condition in the subject, thereby diagnosing the medical condition in the subject.

As used herein the phrase “female reproductive hormone” refers to a hormone secreted by or acting on the female reproduction system, such as hormones secreted by the gonads (ovaries), fatty tissue, adrenal glands, and placenta.

As mentioned, the method is effected by administering into the skin at least one female reproductive hormone. The female reproductive hormone can be a naturally occurring hormone, a synthetic preparation of a hormone or a derivative thereof. Non-limiting examples of female reproductive hormones which can be used by the method of the invention include, estrogens such as estriol [also referred to as oestriol; C₁₈H₂₄O₃; or a derivative thereof such as estriol-3-glycidyl ether, 2-methoxy-estriol, 16-epiestriol], estradiol [17β-estradiol, also referred to as oestradiol; C₁₈H₂₄O₂; or a derivative thereof such as 17 beta estradiol valerate, 2-hydroxy-estradiol], estrone [also called oestrone; C₁₈H₂₂O₂; or a derivative thereof such as estrone sulfate, 2-hydroxy-estrone, 4-hydroxy-estrone, 16α-hydroxy-estrone, 2-methoxy-estrone, 3-epoxypropoxy-estra-1.3.5(10)-trien-17-one], progesterone (or a synthetic hormone of the progestin class, such as progestogen; or a derivative thereof such as pregnenolone or 17-hydroxyprogesterone, medroxyprogesterone acetate].

As used herein the term “subject” refers to a male or female mammal e.g., a human being, of any age.

According to some embodiments of the invention the subject in need of the diagnosis does not have symptoms of or is not being tested for estrogen dermatitis.

Estrogen dermatitis is characterized by eruptions (e.g., papulovesicular eruptions, acneiform eruption), urticaria, eczema, pruritus (e.g., pruritic lesions on face, neck, back, arms and/or legs), prurigo, and/or erythema (e.g., annular erythema), which are caused by or associated with hypersensitivity to estrogen.

The skin of a human body is subdivided into three compartments: an epidermis, a dermis and a subcutaneous layer. As used herein the phrase “into the skin” refers to injection into the epidermis and/or the dermis layers.

It should be noted that the epidermis includes keratinocytes, Langerhans cells, and T-lymphocytes, and is devoid of blood vessels (Kanitakis J. Anatomy, histology and immunohistochemistry of normal human skin. Eur J. Dermatol. 2002 July-August; 12(4):390-9. Review, which is hereby incorporated by reference in its entirety; Spetz A L, Strominger J, Groh-Spies V. T cell subsets in normal human epidermis. Am J Pathol. 1996 August; 149(2):665-74, which is hereby incorporated by reference in its entirety). Thus, administering the female reproductive hormone(s) into the epidermis layer results in a local immune response while preventing a substantial systemic response via the peripheral blood.

Methods of administering an active agent into a skin are known in the art and include, for example, intradermal injections, gels, liquid sprays and patches which comprise the active agent and which are applied on the outer surface of the skin.

According to some embodiments of the invention, administration of the active agent (the at least one female reproductive hormone) into the skin of the subject is performed topically (on the skin).

According to some embodiments of the invention, administration of the active agent (the at least one female reproductive hormone) into the skin of the subject is performed non-invasively, e.g., using a gel, a liquid spray or a patch comprising the active ingredient, which are applied onto the skin of the subject.

There are two main types of skin patches which can be used to administer the female reproductive hormone into the skin of a subject. These are the reservoir type patch and the matrix type patch. The reservoir patch usually contains a structure filled with a solid drug (active agent) and a dilute solution, or a highly concentrated drug solution within a polymer matrix and is surrounded by a film or membrane of rate-controlling material. The matrix patch contains a drug and a polymer which form a homogenous system from which the drug is released by diffusion into the external environment. It should be noted that as the release continues, its rate in the matrix type patch usually decreases since the active agent has a progressively longer distance and therefore requires a longer diffusion time to release. For further details and examples of transdermal drug delivery see Prausnitz M R., et al., 2004. Nature Reviews, 3:115-124; Scheindlin S., 2004. Transdermal drug delivery: Past, present, future. Molecular Interventions. Vol. 4:308-312; Prausnitz M R and Langer R., 2008, Nature Biotechnology. 26:1261-1268; Tanner T, and Marks R, 2008, Delivery drugs by transdermal route: review and comment. Skin Research and Technology, 14: 249-260; each of which is hereby incorporated by reference in its entirety).

A non-limiting example of an epicutaneous drug delivery patch, which can be used to administer the female reproductive hormone into the skin according to the teachings of the invention, is described in Senti G., et al., 2009, J Allergy Clin Immunol. Sep. 4. [Epub ahead of print], which is hereby incorporated by reference in its entirety).

According to some embodiments of the invention, administering the at least one female reproductive hormone to the skin is performed using a reservoir type patch.

According to some embodiments of the invention, administering the at least one female reproductive hormone to the skin is effected on an intact skin (e.g., a skin which has not been breached, peeled or physically/chemically permeabilized).

For example, administering into an intact skin can be performed using an occlusive patch with semi-solid reservoir and a plastic backing adhesive contour and protective removable cover.

A semi-solid reservoir can be any gel, cream, ointment, emulsion, suspension, microparticles, using various excipients such as fats, oils (e.g., mineral oil, vaselin, vegetable oil or silicon oil), polymers, gelling agent, suspending agent, stabilizers, hydrophilic solvents, Propylene glycol, polyethylene glycols, stabilizing surfactants, colloids etc. and their combinations.

It should be noted that in order to increase delivery of the active agent into the skin, the active agent can be formulated with various vehicles designed to increase delivery to the epidermis or the dermis layers. Such vehicles include, but are not limited to liposomes, dendrimers, noisome, transfersome, microemulsion and solid lipid nanoparticles (for further details see Cevc, G. Transfersomes, liposomes and other lipid suspensions on the skin: permeation enhancement, vesicle penetration, and transdermal drug delivery. Crit. Rev. Ther. Drug Carrier Syst. 13, 257-388 (1996), which is hereby incorporated by reference in its entirety; Kogan A, Garti N. Microemulsions as transdermal drug delivery vehicles. Adv Colloid Interface Sci 2006; 123-126:369-385, which is hereby incorporated by reference in its entirety). In addition, the active agent can be mixed with chemical enhancers such as sulphoxides, azones, glycols, alkanols and terpenes which enhance delivery of active agents into the skin (for further details see Karande P, Jain A, Ergun K, Kispersky V, Mitragotri S. Design principles of chemical penetration enhancers for transdermal drug delivery. Proc Natl Acad Sci US A 2005; 102:4688-4693; Williams A C, Barry B W. Penetration enhancers. Adv Drug Deliv Rev 2004; 56:603-618; and Smith, E W.; Maibach, H I., editors. Boca Raton, Fla.: Taylor and Francis Group; 2006. Percutaneous Penetration Enhancers; each of which is hereby incorporated by reference in its entirety).

The patch may include the female reproductive hormone formulated within an emulsion designed to facilitate permeabilization of drugs to the epidermis or the dermis. For example, the patch may comprise the female reproductive hormone within an oil-in-glycerin emulsion, which is designed to facilitate permeabilization of the female reproductive hormone through the stratum-corneum and into the dermis. A non-limiting example of an oil-in-glycerin emulsion suitable for delivery through the stratum-corneum into the dermis is described in US Patent Application No. 20040067244, which is hereby incorporated by reference in its entirety. Such an oil-in-glycerin emulsion exhibits a mean droplet size below one micron, and comprises a continuous glycerin phase; at least one vegetable oil comprising an internal phase; at least one emulsifying stabilizer; and at least one bioactive compound comprising at least one hydrophobic, moiety within its structure, wherein the composition facilitates permeabilization of the bioactive compound through the stratum-corneum and into the dermis.

According to some embodiments of the invention, administering the at least one female reproductive hormone to the skin is effected on a breached skin [e.g., a skin that has been permeabilized (e.g., ruptured) with an external object and the like].

According to some embodiments of the invention, breaching of the skin is effected temporarily (e.g., performed for a pre-determined short period) and is designed to enable better permeabilization of the active ingredient into the skin.

Breaching of the skin can be performed, for example, by introducing micro-holes (e.g., microchannels) in the outer layer of the skin. Such microchannels can be formed using for example, the Radio-Frequency (RF)-Microchannel™ (TransPharma Medical™ Ltd.) technology [Hypertext Transfer Protocol ://World Wide Web (dot) transpharma-medical (dot) com/technology_rf (dot) html].

Additionally or alternatively, delivery of the active agent (e.g., the female reproductive hormone) from the patch to the epidermis layer of the skin can be enhanced using physical enhancers known in the art such as ultrasound, ionophoresis, electroporation, magnetophoresis, microneedle and continuous mixing [see e.g., Rizwan M, Aqil M, Talegaonkar S, Azeem A, Sultana Y, Ali A. Enhanced transdermal drug delivery techniques: an extensive review of patents. Recent Pat Drug Deliv Formul. 2009; 3(2):105-24, which is hereby incorporated by reference in its entirety].

According to some embodiments of the invention, administering the female reproductive hormone is performed by an intradermal injection.

The female reproductive hormone can be administered into the dermal layer of the skin of the subject by an intradermal injection as described for the Mantoux C (1908) test. Briefly, the female reproductive hormone can be injected intracutaneously (using for example, a 0.5-ml or 1.0 ml tuberculin syringe through a 26-gauge or 27-gauge needle). The syringe can be placed at an angle of 45 degrees to the skin, and the bevel of the needle is angled downward, facing the skin, and penetrating entirely but not deeper than the superficial layers of the skin. A volume of approximately 0.01 to 0.05 ml (e.g., about 0.02 ml) is gently injected to produce a small superficial bleb (Middleton's Allergy principles&practice, 6^{th} edition 2003).

According to some embodiments of the invention, administering the female reproductive hormone is performed using a liquid spray (e.g., a spray which includes the female reproductive hormone in a pre-determined concentration and dosage).

According to some embodiments of the invention, administering the female reproductive hormone is performed using a gel (e.g., a gel which includes the female reproductive hormone in a pre-determined concentration and dosage).

For example, for administration using a gel or a spray, a predefined area for administration of the hormone is selected and optionally bounded using an accessory equipment (see e.g., Hypertext Transfer Protocol://World Wide Web (dot) truetest (dot) com).

According to some embodiments of the invention, administering the at least one female reproductive hormone to the skin is effected such that the required hormone dose is delivered to the skin epidermis and/or dermis layers within a short time, mimicking the effect of an injection into the epidermis or dermis layers.

The method according to some embodiments thereof is effected by administering into the skin two or more of the female reproductive hormones such as estriol and estradiol; estriol and estrone; estriol and progesterone; estrone and progesterone; estrone and estradiol; estriol, estradiol and progesterone; estriol, estrone and progesterone; estriol, estradiol and estrone; estrone, estradiol and progesterone; estriol, estradiol, estrone and progesterone.

As mentioned, the female reproductive hormone can be formulated for administration into the skin via an injection, a gel, a liquid spray and/or a patch.

According to some embodiments of the invention the active ingredients (e.g., in the formulation) may comprise a combination of one or more of the female reproductive hormones. For example, the active ingredients may comprise a combination of estrogens (e.g., estriol and estradiol; estriol and estrone; estrone and estradiol; estriol, estradiol and estrone). Additionally or alternatively, the active ingredients (e.g., in the formulation) may comprise progesterone. Additionally or alternatively, the active ingredients (e.g., in the formulation) may comprise a combination of estrogen (e.g., estriol, estradiol or estrone) and progesterone.

The dose of the female reproductive hormone which is administered into the skin of the subject is selected such that is induces a reaction on the skin of the subject.

As used herein “a reaction” refers to a change of color, texture or swelling of the skin.

According to some embodiments of the invention, injection to the skin (e.g., an intradermal injection) of the subject is effected at a dose of at least about 0.1 μg, at least about 0.2 μg, at least about 0.3 μg, at least about 0.4 μg, at least about 0.5 μg, at least about 1 μg, at least about 2 μg, at least about 3 μg, at least about 4 μg, at least about 5 μg, at least about 6 μg, at least about 7 μg, at least about 8 μg, at least about 9 μg, at least about 10 μg, at least about 12 μg, at least about 15 μg, at least about 20 μg, at least about 25 μg, at least about 30 μg, at least about 40 μg, at least about 45 μg, at least about 50 μg, at least about 55 μg at least about 60 μg, at least about 70 μg, at least about 80 μg, at least about 90 μg, at least about 100 μg, at least about 200 μg, at least about 500 μg, at least about 1000 μg, at least about 2 milligram (mg).

According to some embodiments of the invention, injection to the skin (e.g., an intradermal injection) of the subject is effected at a dose in the range of about 0.1 μg to about 200 μg, e.g., about 1 μg to about 100 μg, e.g., about 1 μg to about 60 μg, e.g., about 1 μg to about 50 μg, e.g., about 2 μg to about 40 μg, e.g., about 2 μg to about 30 μg, e.g., about 2 μg to about 20 μg, e.g., about 10 μg.

According to some embodiments of the invention, administering to the skin of the subject is effected using a patch, a gel or a spray at a dose of at least about 0.1 μg per 1 cm² area, at least about 0.2 μg per 1 cm² area, at least about 0.3 μg per 1 cm² area, at least about 0.4 μg per 1 cm² area, at least about 0.5 μg per 1 cm² area, at least about 1 μg per 1 cm² area, at least about 2 μg per 1 cm² area, at least about 3 μg per 1 cm² area, at least about 4 μg per 1 cm² area, at least about 5 μg per 1 cm² area, at least about 6 μg per 1 cm² area, at least about 7 μg per 1 cm² area, at least about 8 μg per 1 cm² area, at least about 9 μg per 1 cm² area, at least about 10 μg per 1 cm² area, at least about 12 μg per 1 cm² area, at least about 15 μg per 1 cm² area, at least about 20 μg per 1 cm² area, at least about 25 μg per 1 cm² area, at least about 30 μg per 1 cm² area, at least about 40 μg per 1 cm² area, at least about 45 μg per 1 cm² area, at least about 50 μg per 1 cm² area, at least about 55 μg per 1 cm² area, at least about 60 μg per 1 cm² area, at least about 70 μg per 1 cm² area, at least about 80 μg per 1 cm² area, at least about 90 μg per 1 cm² area, at least about 100 μg per 1 cm² area, at least about 200 μg per 1 cm² area, at least about 500 μg per 1 cm² area, at least about 1000 μg per 1 cm² area, at least about 2 milligram (mg) per 1 cm² area, e.g., about 10 μg per 1 cm² area.

According to some embodiments of the invention, administering to the skin of the subject is effected using a patch, a gel or a spray at a dose in the range of about 0.1 μg per 1 cm² area to about 200 μg per 1 cm² area, e.g., about 1 μg per 1 cm² area to about 100 μg per 1 cm² area, e.g., about 1 μg per 1 cm² area to about 60 μg per 1 cm² area, e.g., about 1 μg per 1 cm² area to about 50 μg per 1 cm² area, e.g., about 2 μg per 1 cm² area to about 40 μg per 1 cm² area, e.g., about 2 μg per 1 cm² area to about 30 μg per 1 cm² area, e.g., about 2 μg per 1 cm² area to about 20 μg per 1 cm² area.

According to some embodiments of the invention, the patch which comprises the at least one female reproductive hormone exhibits a size of about 0.5 cm² to about 10 cm², e.g., about 0.6 cm² to about 8 cm², e.g., about 0.8 cm² to about 6 cm², e.g., about 1 cm² to about 5 cm², e.g., about 1 cm² to about 4 cm², e.g., about 1 cm² to about 2 cm².

According to some embodiments of the invention, administering using a patch, a gel or a spray is effected for a time period of about 1 minutes to about 72 hours, e.g., from about 10 minutes to about 72 hours, e.g., from about 30 minutes to about 60 hours, e.g., from about 1 hour to about 48 hours, e.g., from about 2 hours to about 45 hours, e.g., from about 3 hours to about 40 hours, e.g., from about 5 hours to about 30 hours, e.g., from about 10 hours to about 24 hours, e.g., from about 12 hours to about 24 hours, e.g., from about 12 hours to about 20 hours, e.g., from about 12 hours to about 15 hours, e.g., for about 1 hour.

The female reproductive hormone can be administered to any site or portion of the subject's skin.

According to some embodiments of the invention, the female reproductive hormone is administered to the buttock, the abdomen, upper outer arm, upper torso (excluding breasts) and/or the thigh of the subject in need thereof (e.g., which is diagnosed and/or treated according to the method of the invention).

According to some embodiments of the invention, the dose of the female reproductive hormone is selected such that it does not cause eruption of symptoms associated with the medical condition (e.g., does not exceed 20 mg, see e.g., Hobbart W. Walling et. al. Histological overlap of erythema multiforme and urticaria. Journal of Dermatology 2008, 47, 380-382).

The following exemplary doses are provided for administration into the skin for diagnostically monitoring hypersensitivity: 17 beta estradiol valerate (E2) from about 3.6 μg to about 21 μg; estrone (E1) from about 2.7 μg to about 16 μg; estriol (E3) from about 8.7 μg to about 52 μg; progesterone (P) from about 3.1 μg to about 19 μg.

As shown in Table 2 in Example 1 of the Examples section which follows, the female reproductive hormone can be dissolved in a diluent.

According to some embodiments of the invention, the diluent is biocompatible, thus, when in contact with cells, tissues or body fluid of a subject does not induce adverse effects such as a pronounced immunological reaction. Non-limiting examples of biocompatible diluents include saline, a biocompatible oil, alcohol and the like.

According to some embodiments of the invention, the diluent comprises an oil such as a synthetic oil, mineral oil, ethyl oleate, a vegetable or fruit oil such as Soy-bean oil, groundnut oil, sesame oil, peach oil, peanut oil, almond oil and the like, an animal oil or a combination thereof.

According to some embodiments of the invention, the diluent comprises an alcohol or an alcohol solution. Non-limiting examples of suitable diluents include ethyl alcohol, and benzyl alcohol.

According to some embodiments of the invention, the diluent further comprises an enhancer for increasing permeabilization of the skin to the female reproductive hormone.

According to some embodiments of the invention, when using a matrix patch the diluent of the hormone further comprises additives such as enhancers and adhesives.

The volume of the diluent, which is used for dissolving the hormone, is selected depending on the administration route (e.g., injection, matrix patch, reservoir patch, gel, spray and the like) and those of skills in the art are capable of adjusting the diluent volume to the administration route.

According to some embodiments of the invention the diluent's viscosity at room temperature is selected for smooth intradermal introduction. Adjustment of the viscosity can be performed by adding a solvent such as ethyl or benzyl alcohol to the hormone's diluent. The final concentration of the solvent (e.g., benzyl alcohol, ethyl alcohol) within the diluent can be in the range of about 5-45%.

According to some embodiments of the invention, the hormone is diluted in a volume of about 0.1 ml to about 1.5 ml of the diluent.

Monitoring the reaction to the female reproductive hormone can be performed by evaluating presence/absence and/or size (dimensions) of a wheal formed in response to the hormone. The reaction can be characterized by a change of skin color (e.g., to a more pink or red color), texture and/or by swelling of the skin.

For example, monitoring the reaction on the skin (the wheal) can be performed using an image analysis device or apparatus which analyzes the reaction's parameter(s) such as the length, diameter, area, volume, height, degree of color, shape, shades and/or heat of the reaction. Such parameters can be extracted from an image of the reaction on the skin (the wheal), which can be captured using known means such as a still or video camera (e.g., a digital camera).

According to some embodiments of the invention the size of the wheal is determined by the average wheal's diameter which can be calculated by averaging the longest diameter of the wheal and the perpendicular diameter thereof according to formula I.

wheal diameter=(D+d)/2 Formula I:

where D is the maximum diameter and d is perpendicular diameter.

According to some embodiments of the invention the dimensions of the wheal are determined by the average wheal's area or volume.

According to some embodiments the diluent per se is used as the test control.

As used herein, the phrase “above a predetermined threshold” refers to the difference in the reaction on the skin (e.g., as determined by the average diameter of the wheal, the area of the wheal or the volume of the wheal) to the administered hormone as compared to the reaction generated by the test control.

The threshold can be a difference of at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 11 mm, at least about 12 mm, at least about 13 mm, at least about 14 mm, at least about 15 mm in the diameter of the wheal in reaction to the administered hormone as compared to the test control.

Additionally or alternatively, the threshold can be a difference of at least about 0.7 mm², at least about 1 mm², at least about 2 mm², at least about 3 mm², at least about 4 mm², at least about 5 mm², at least about 6 mm², at least about 7 mm², at least about 8 mm², at least about 9 mm², at least about 10 mm², at least about 11 mm², at least about 12 mm², at least about 13 mm², at least about 14 mm², at least about 14 mm², at least about 15 mm², at least about 16 mm², at least about 20 mm², at least about 30 mm², at least about 40 mm², at least about 50 mm², at least about 60 mm², at least about 70 mm², at least about 80 mm², at least about 90 mm², at least about 100 mm², at least about 110 mm², at least about 120 mm², at least about 130 mm², at least about 140 mm², at least about 150 mm², at least about 160 mm², at least about 170 mm² in the area of the wheal in reaction to the administered hormone as compared to the test control.

For example, the threshold can be a difference of about 0.8 mm², about 3.1 mm², about 7.1 mm², about 12.6 mm², about 19.6 mm², about 28.3 mm², about 38.5 mm², about 50.2 mm², about 63.6 mm², about 78.5 mm², about 95.0 mm², about 113.0 mm², about 132.7 mm², about 153.9 mm², about 176.6 mm² in the area of the wheal in reaction to the administered hormone as compared to the test control.

Additionally or alternatively, the threshold can be a difference of at least about 0.26 mm³, at least about 1 mm³, at least about 2 mm³, at least about 3 mm³, at least about 4 mm³, at least about 5 mm³, at least about 6 mm³, at least about 7 mm³, at least about 8 mm³, at least about 9 mm³, at least about 10 mm³, at least about 11 mm³, at least about 12 mm³, at least about 13 mm³, at least about 14 mm³, at least about 15 mm³, at least about 14 mm³, at least about 15 mm³, at least about 16 mm³, at least about 20 mm³, at least about 30 mm³, at least about 40 mm³, at least about 50 mm³, at least about 60 mm³, at least about 70 mm³, at least about 80 mm³, at least about 90 mm³, at least about 100 mm³, at least about 110 mm³, at least about 120 mm³, at least about 130 mm³, at least about 140 mm³, at least about 150 mm³, at least about 160 mm³, at least about 170 mm³ in the volume of the wheal in reaction to the administered hormone as compared to the test control.

For example, the threshold can be a difference of about 0.26 mm³, about 0.52 mm³, about 1.05 mm³, about 2.09 mm³, about 2.36 mm³, about 4.19 mm³, about 4.71 mm³, about 6.54 mm³, about 8.37 mm³, about 9.42 mm³, about 12.82 mm³, about 13.08 mm³, about 16.75 mm³, about 18.84 mm³, about 21.20 mm³, about 25.64 mm³, about 26.17 mm³, about 31.66 mm³, about 33.49 mm³, about 37.68 mm³, about 42.39 mm³, about 44.22 mm³, about 51.29 mm³, about 52.33 mm³, about 58.88 mm³, about 63.32 mm³, about 75.36 mm³, about 88.44 mm³, about 102.57 mm³, about 117.75 mm³ in the volume of the wheal in reaction to the administered hormone as compared to the test control

A difference above a predetermined threshold enables detection of the hypersensitivity to the hormone rather than to the hormone diluent (the negative control solution).

According to some embodiments of the invention, the predetermined threshold comprises an increase in diameter of at least 5 mm in the reaction as compared to control test.

Of note, a difference of, or above 20 mm in diameter may be indicative of medical conditions other than hypersensitivity such as an acute phases autoimmune disease or a malignancy.

In addition, a difference of, or above 314 mm² in the area or 209 mm³ in the volume of the wheal may be indicative of medical conditions other than hypersensitivity such as an acute phases autoimmune disease or a malignancy.

Monitoring of the skin reaction can be effected daily (or every other day, three days, four days, 5 days, 6 days, weekly, etc.), for at least 1 day, e.g., at least 2 days, e.g., at least 3 days, e.g., at least 4 days, e.g., at least 5 days, e.g., at least 6 days, e.g., at least 7 days, e.g., at least 8 days, e.g., at least 9 days, e.g., at least 10 days, e.g., at least 11 days, e.g., at least 7 days, e.g., at least 8 days, e.g., at least 9 days, e.g., at least 10 days, e.g., at least 11 days, e.g., at least 12 days, e.g., at least 13 days, e.g., at least 14 days, e.g., at least 15 days, e.g., at least 16 days, e.g., at least 17 days, e.g., at least 18 days, e.g., at least 19 days, e.g., at least 20 days, e.g., at least 21 days, e.g., at least 22 days, e.g., at least 23 days, e.g., at least 24 days, e.g., at least 25 days, e.g., at least 26 days, e.g., at least 27 days, e.g., at least 28 days, e.g., at least 29 days, e.g., at least 30 days.

According to some embodiments of the invention, monitoring of the skin reaction is effected following 7 days.

According to some embodiments of the invention, monitoring of the skin reaction is effected following a complete menstrual cycle or 30 days.

As mentioned, presence of the reaction above the predetermined threshold is indicative of the presence of the medical condition in the subject.

According to some embodiments of the invention, the medical condition which is diagnosed by the method of this aspect of the invention is premenstrual syndrome (PMS), which symptoms include, but are not limited to, acne, anger, anxiety, appetite shift, blind spots, bloating, cramps, crave for sweet, crying, chest pains, cold sweats, depression, decreased efficiency, dizziness, fuzzy vision, forgetfulness, faintness, feeling of suffocation ness, general pain, heart pounding, headache, hot flashes, insomnia, itch, irritability, leg swell, low back pain, mastalgia, mood shifts, muscle stiffness, nausea, numbness, ringing in the ears, stress, stomach ache, swelling, tingling tiredness, vomiting, water retention and weight gain or various combinations thereof.

According to some embodiments of the invention, the medical condition which is diagnosed by the method of this aspect of the invention is characterized by any of the following symptoms or various combinations thereof, even when not-related to the menstrual cycle (e.g., when the symptoms do not change during the menstrual cycle or when the symptoms are present in non-fertile females such as females in menopause) acne, asthma, anger, anxiety, appetite shift, bloating, cramps, crave for sweet, crying, chronic fatigue syndrome, coronary events sympathovagal cardial accidence (syncope, paroxysmal tachycardia, hypotension and or/hypertension), climacteric disorders possessed altered sympathovagal balance (hot flashes and night sweats), diabetes (type 2), depression, fibromyalgia, general pain, headache, hot flashes, insomnia, itch, leg swell, low back pain, mastalgia, mood shifts, stress, stomach ache and tiredness.

According to some embodiments of the invention, the medical condition which is diagnosed by the method of this aspect of the invention is premenstrual dysphoric disorder (PMDD); pruritus vulvae; menstrual asthma; menstrual migraine; infertility; premenopausal syndrome (characterized by hot flashes, night sweats, and inconsistent menstruation); menopausal syndrome (characterized by hot flashes and night sweats); recurrent abortions; fatigue syndrome; unexplained preterm delivery; unexplained irritable bowel syndrome; endometriosis; idiopathic premature delivery; sympathovagal dysbalance in pregnancy (hypotension and/or hypertension); menstrual cycle related alterations in metabolic diseases (e.g. Diabetes); hyper-emesis gravidarum; idiopathic gingivitis; unexplained infertility; unexplained recurrent in vitro fertilization (IVF) failure; intrauterine insemination (IUI) failure; IVF or IUI side effects (e.g. ovarian hyperstimulation); unexplained male infertility; hypomagnesemia; menstrual related catamenial epilepsy; menstrual related or unexplained pain conditions (e.g., mastalgia, fibromyalgia, dysmenorrhea and migraine); cognitive sensory and emotional changes associated with the menstrual cycle such as hyperosmia, accidents, confusion, weight gain; premature and unexplained ovarian failure in a subject.

According to some embodiments of the invention, the medical condition which is diagnosed by the method of the invention is not estrogen dermatitis.

In addition, the present inventors have further envisaged the use of the diagnostic test of hypersensitivity to the female reproductive hormone in order to coordinate the time of providing anti-hormonal therapies to a subject. For example, as described in Example 4, anti-hormonal therapy such as anti-estrogen [Tamoxifen 10 (GENERICS LTD, ENGLAND)] or anti-progesterone [Mifepristone, known as RU-486 (Roussel Uclaf)] which is usually given a regular daily schedule and is associated with side effects. The present inventors have further envisaged that these side effects can be minimized or completely disappear if the anti-hormonal therapy is provided on the days the subject exhibits the hypersensitivity to the female reproductive hormone. Similarly, treatment of headaches which are associated with the menstrual cycle can be restricted to the days in which the subject exhibits hypersensitivity to hormones. Thus, the diagnostic method can be used for a timely-coordinated individual therapy, adjusted according to the level of hypersensitivity to hormones as measured in the subject.

In addition, the present inventors have further envisaged the use of the diagnostic test of hypersensitivity to the female reproductive hormone in order to determine the suitability of a subject to a female reproductive hormone therapy such as birth control pills. According to the method, an absence of such a hypersensitivity is indicative of the suitability of the subject to the hormonal therapy. On the other hand, presence of a hypersensitivity to the hormone is indicative of the subject being not suitable for the hormonal therapy.

The present inventors have shown that once a subject is diagnosed with a hypersensitivity to the female reproductive hormone, treatment of the medical condition can be achieved by desensitization of the hypersensitivity.

Thus, as shown in Tables 5-8 and Examples 2 and 3 of the Example section which follows, a desensitization treatment of subjects suffering from PMS was found to be far more efficient in reducing duration and severity of symptoms as compared to conventional therapies. In addition, as shown in Table 9 and described in Example 5 of the Examples section which follows subjects diagnosed with PMS, infertility (e.g., recurrent abortions), premenopausal syndrome or menopausal syndrome (characterized by hot flashes and night sweats) were successfully treated by a desensitization treatment (e.g., abolishment or reduction of severity of PMS or menopausal symptoms and successful pregnancies and newborn deliveries).

Thus, according to an aspect of some embodiments of the invention, there is provided a method of treating a medical condition associated with a hypersensitivity to a female reproductive hormone in a subject. The method is effected by (a) diagnosing the medical condition according to the method of the invention and (b) subjecting the subject to a desensitization treatment to the hypersensitivity to the female reproductive hormone.

The term “treating” as used herein refers to inhibiting, preventing or arresting the development of a disease, disorder or medical condition and/or causing the reduction, remission, or regression of a disease, disorder or medical condition in an individual suffering from, or diagnosed with, the disease, disorder or medical condition. Those of skill in the art will understand that various methodologies and assays can be used to assess the development of a disease, disorder or medical condition, and similarly, various methodologies and assays may be used to assess the reduction, remission or regression of a disease, disorder or medical condition.

The desensitization treatment according to the method of the invention is based on administering into the skin of the subject the female reproductive hormone which the subject is hypersensitive thereto in doses and frequencies that are capable of reducing the hypersensitivity.

As mentioned, according to specific embodiments, administration of the female reproductive hormone to the epidermis or dermis layers of the skin can be performed using an injection, via application of a patch (with or without physical and chemical enhancers as described above), using a gel or a spray.

According to some embodiments of the invention, the desensitization treatment is effected by at least one, at least two, at least three consecutive administrations (e.g., injections or patch-mediated administrations) of the female reproductive hormone into the skin (e.g., into the epidermis and/or intradermally) of the subject, optionally, until relieve of symptoms and/or absence of hypersensitivity to the female reproductive hormone is achieved.

According to some embodiments of the invention, treatment by desensitization is effected using a female reproductive hormone at a concentration of about 0.1 μg to about 1600 μg.

According to some embodiments of the invention, patch mediated desensitization treatment is effected using a female reproductive hormone at a concentration of about 0.8 μg per about 1-30 cm² to about 1600 μg per about 1-30 cm² area.

According to some embodiments of the invention, the female reproductive hormone is estradiol, estrone and/or progesterone, and the first administration includes a dose of about 0.1-25 μg of the female reproductive hormone.

According to some embodiments of the invention, the female reproductive hormone is estriol, and the first administration includes a dose of about 0.1-55 μg of the female reproductive hormone.

According to some embodiments of the invention, the dose of female reproductive hormone used for desensitization treatment does not exceed the maximum dose of skin test as described above.

According to some embodiments of the invention, each consecutive administration includes a dose of the hormone which is identical or higher than the previous administration.

According to some embodiments of the invention, the desensitization treatment is effected by three consecutive administrations, in which the first administration is effected at a dosage of about 0.1-55 μg of the female reproductive hormone, e.g., about 0.1-50 μg of the female reproductive hormone, the second administration is effected at an identical or larger dosage of the first administration and the third administration is effected at an identical or larger dosage of the second administration.

According to some embodiments of the invention, the desensitization treatment is effected by three consecutive administration, in which the first administration is effected at a dosage of about 0.1-55 μg of the female reproductive hormone, e.g., about 0.1-50 μg of the female reproductive hormone, the second administration is effected at a dose which is twice the dosage of the first administration and the third administration is effected at a dosage which is twice the dosage of the second administration.

A non-limiting example for the desensitization treatment according to the method of the invention is injecting about 5.4 μg of estriol in the first administration; about 10.8 μg of estriol in the second administration, and about 21.6 μg of estriol in the third administration. In addition, the desensitization treatment may also include another female reproductive hormone (for which the subject exhibits hypersensitivity) such as estradiol. Thus, the desensitization treatment may also include injecting about 7.1 μg of 17 beta estradiol valerate in the first administration; about 14.2 μg of 17 beta estradiol valerate in the second administration, and about 28.2 μg of 17 beta estradiol valerate in the third administration.

According to some embodiments of the invention, the patch mediated desensitization treatment is effected by three consecutive administrations (e.g., using three patches), in which the first patch comprises a dosage of about 20 μg of the female reproductive hormone in a 2-cm² area patch, the second patch comprises a dosage of about 40 μg of the female reproductive hormone in a 4-cm² area patch, and the third patch comprises a dosage of about 80 μg of the female reproductive hormone in an 8-cm² area patch.

According to some embodiments of the invention, the intervals between the various desensitization administrations depend on the fertility state of the subject. Such intervals can be selected from the range of one day to a whole month (or a complete menstrual cycle).

The normal length of the menstrual cycle in the reproductive age varies from 25 to 35 days and includes a luteal phase of 13-16 days. Most fertile females have a menstrual cycle of 27 to 31 days with regular ovulation.

Methods of documenting ovulation are known in the art and include, for example, detection of luteinizing hormone, detection of basal body temperature, mid-luteal serum progesterone, ultrasound monitoring ovulation (by monitoring the development of a dominant follicle), presence of premenstrual breast swelling and dysmenorrhea (Berek & Novak's Gynecology, Fourteenth Edition 2007).

A female is considered “non-fertile” if being in menopause or having an irregular unovulatory menstrual cycle, women with hypothyroidism, anorexia nervosa, polycystic ovarian syndrome (PCOS), oligo-ovulation (infertile) and premature ovarian failure [ovarian failure is a normal occurrence during menopause]. Premature ovarian failure occurs before 40 years of age in 1% to 5% of women and is considered pathologic.

Menopause is defined retrospectively at the time of the final menstrual period followed by 12 months of amenorrhea (Berek &Novak's Gynecology, Fourteenth Edition 2007. (The age of menopause is determined by genetic inheritance.

According to some embodiments of the invention, when treating a fertile female the interval(s) between the three consecutive administrations can be of a complete menstrual cycle.

According to some embodiments of the invention, when the subject is fertile, the first administration of the desensitization treatment can be during the luteal phase of the menstrual cycle.

For example, when the subject is considered a priori fertile, the first administration can be effected during a luteal phase of the menstrual cycle, the second administration is effected following a complete menstrual cycle, and a third administration is effected following a complete menstrual cycle of the second administration.

According to some embodiments of the invention, when the subject is non-fertile, e.g., in menopause, or in the reproductive age (e.g., 16-45 years old) but having an irregular menstrual cycle or an unovulation, the interval(s) between the three consecutive administrations are of one week.

Alternatively, the intervals between the three administrations can be of one week, regardless of the fertility state of the subject.

As shown in Table 4 and described in Example 2 of the Examples section which follows, after desensitization with the hormone, skin reaction and symptom(s) severity were tracked daily to assess treatment effect on hypersensitivity and clinical symptoms and suitability of the subject to a subsequent treatment.

According to some embodiments of the invention, following the first administration and optionally before the scheduled next administration the effect of the treatment on the hypersensitivity of the subject to the hormone and/or on the symptoms of the medical condition is evaluated and continuation to the next desensitization administration is considered based on the effect of treatment.

The effect of the treatment on the medical condition symptoms is optionally determined by taking into consideration the most dominant symptom or by averaging several symptoms using known algorithms (for example, averaging the VAS score in case of PMS).

For example, if following the first desensitization treatment the hypersensitivity to the female reproductive hormone is reduced (e.g., the difference in the diameter of the reaction on the skin to the hormone is less than 5 mm as compared to the reaction on the skin to the diluent of the hormone) or completely disappears (e.g., no skin reaction at all), and/or if the subject is completely or substantially free of symptoms of the medical condition, no further desensitization administrations are required.

Alternatively, if following the first desensitization treatment a moderate reduction or no reduction in the hypersensitivity and/or symptoms is seen, then the desensitization treatment is continued with a subsequent administration.

Similarly, if following the first desensitization treatment the hypersensitivity to the hormone is increased or not changed (as determined by the skin test) but the symptoms of the medical condition are reduced, the desensitization treatment is continued with the subsequent administration.

If following the first desensitization treatment the hypersensitivity is decreased (as determined by the skin test) but the symptoms of the medical condition are not changed, the desensitization treatment continues with the subsequent administration, optionally with more than 3 administrations altogether (e.g., 4 or 5 administrations).

On the other hand, if following the first desensitization treatment the symptom(s) of the medical condition increase (i.e., become more severe), then according to some embodiments of the invention a subsequent desensitization treatment is not recommended.

Similar evaluation of the effect of treatment on the subject's hypersensitivity and/or symptoms can be performed after each administration of the desensitization treatment.

According to some embodiments of the invention, such an evaluation is performed following one week or a complete menstrual cycle from the previous administration.

Alternatively, if following the third administration of the desensitization treatment the hypersensitivity is reduced (but not completely disappear) and/or the severity of symptoms is reduced, but not completely disappear, the continuation of treatment to subsequent administrations is optional.

Thus, the desensitization treatment of the subject can be adjusted based on the skin test results of the subject before treatment begins (e.g., when the subject is diagnosed as having a hypersensitivity to the hormone), and based on the hypersensitivity and change in clinical symptoms after each desensitization administration (a personalized treatment).

According to some embodiments of the invention, the desensitization treatment of the invention can be supplemented with magnesium which is provided to the subject at the time the subject exhibits hypersensitivity to the hormone. The magnesium can be applied locally as a gel preparation (at the site of skin reaction) or systemically, at a dosage of 10-30 mg/kg of subject's body/day.

Non-limiting examples of magnesium supplements which can be used by the method of the invention include magnesium tablets (e.g., “Anti Leg Cramps” by Nave Pharm, Israel, which also includes 10 mg Vitamin B and 50 mg Vitamin E); a magnesium patch; a magnesium cream or a magnesium gel.

The present inventors have further envisaged the use of magnesium supplements given to the subject at the time the subject exhibits hypersensitivity to the female reproductive hormone (as detected by the diagnostic test described above) even without the desensitization treatment, e.g., as an independent treatment or along with other (not desensitization) treatment regimens.

The teachings of the invention can be implemented into a computing platform for designing a treatment regimen against a hypersensitivity of a subject to a female reproductive hormone.

As used herein the phrase “treatment regimen” refers to a treatment plan that specifies the type of treatment, dosage, schedule and/or duration of a treatment provided to a subject in need thereof (e.g., a subject diagnosed with the hypersensitivity to the female reproductive hormone). Examples of treatments (e.g., for PMS) include but are not limited to, desensitization treatment (such as described above), analgesics, antidepressant drugs, anti-anxiety drugs, ovulation suppression drugs, magnesium supplements and herbal supplement (Agnus Castus).

Thus, according to an aspect of some embodiments of the invention there is provided a computing platform for designing a treatment regimen against hypersensitivity to a female reproductive hormone.

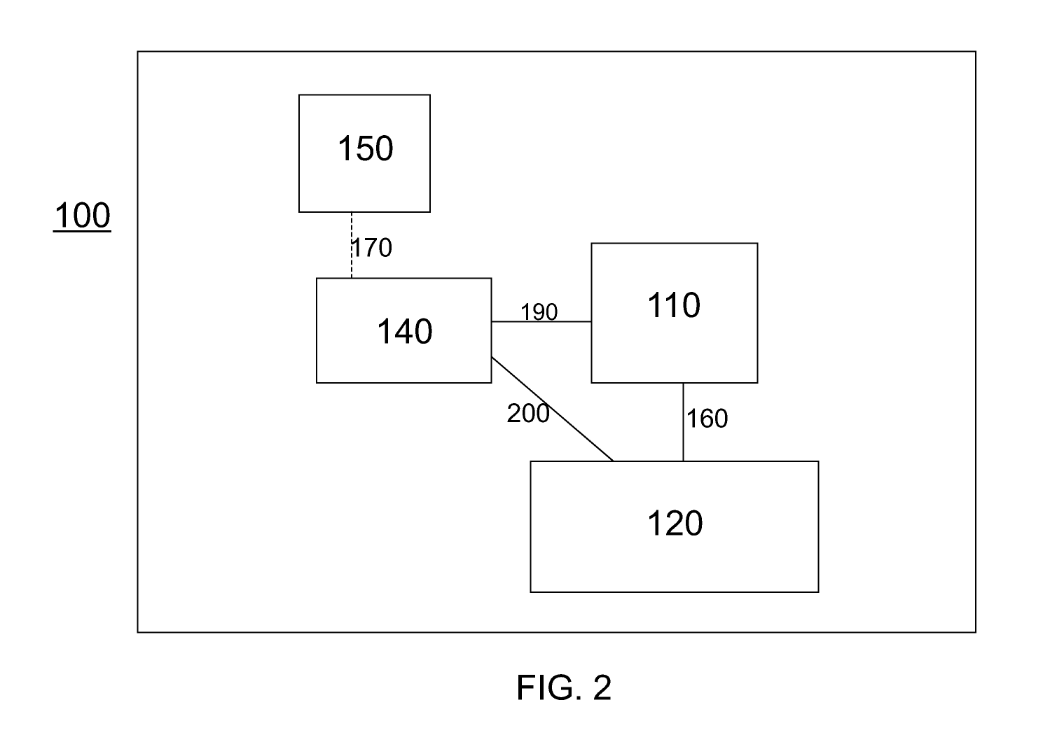

According to some embodiments of the invention a specific configuration of the computing platform is provided in FIG. 2 which shows system 100. System 100 comprises data storage device 110 for storing clinical data of the subject including skin test results against the female reproductive hormone; processing unit 120 being for diagnosing hypersensitivity to a female reproductive hormone and/or designing treatment regimen against the hypersensitivity to the female reproductive hormone using the data stored on the data storage devise; and user interface 140 for displaying patient's diagnosis and/or the treatment regimen of the subject.

System 100 may be any computing platform known in the art including but not limited to personal computer, a work station, a mainframe and the like.

According to some embodiments of the invention, system 100 is provided with a kit or a code to access a web site. The web site can send the user reminders regarding various steps of the diagnostic and/or treatment method (e.g., reminders to test the reaction on the skin after the skin test and/or the desensitization). Optionally, each kit or code for web site has different dosages for the skin test or the desensitization treatment.

Data storage device 110 can be a computer readable medium which can be used to store the skin test results including, but not limited to, hard drives, floppy disks, DAT tape, CD-ROM and other magnetic, magneto-optical, optical, floptical and other media which may be adapted for use with system 100.

According to some embodiments of the invention, the data stored on data storage device 110 further includes demographic data, anamnesis data associated with the hypersensitivity to the female reproductive hormone. Examples include, but are not limited to, body mass index (BMI) (see Example 2 of the Examples section which follows for an exemplary effect of BMI on the desensitization treatment), birth weight [e.g., the more severe symptom(s) is correlated with low birth weight], regularity of menstrual cycle (as described hereinabove and in Example 2 which follows) and gestational age at birth; as well as state of the medical conditions symptoms (e.g., using the VAS scoring system).

According to some embodiments of the invention, the data stored on data storage device 110 further includes subject's general sense of well being [e.g., mental state, physical parameters, blood tests, electrocardiography (ECG or EKG), vital signs, mood, personal feelings, quality of life].

Processing unit 120 can be a computing unit for executing the method of treating the medical condition according to the invention. The algorithm which executes the method receives an input data (anamnesis data, results of hypersensitivity test, and optionally state of symptoms of the medical condition) and produces an output data of a treatment regimen (types of hormones, dosages, intervals, combination with other treatments, supplementation with magnesium, and the like). The algorithm uses a set of rules such as described hereinabove and in the Examples section which follows, in which the skin test results along with the anamnesis data and state of symptoms determine the treatment regimen of the subject. Processing unit 120 is functionally connected to data storage device 110 by connection 160 described below. The connection may be bi-directional. Likewise processing unit 120 and user interface 140 also share a two-way communication channel (connection 200).

User interface 140 can be used for inputting the data and for providing output information (e.g., the treating regimen) to the user. User interface 140 can be a keyboard, a mouse, a monitor and the like. User interface 140 can be connected to [via connection 190 (which may be bi-directional), described below] data storage device 110.

According to some embodiments of the invention, user interface 140 can comprise or be connected [e.g., via connection 170 (which may be bi-directional)] to recorder 150. Recorder 150 is used to record the reaction on the skin to the female reproductive hormone (skin test results), the symptoms of the medical condition and/or the subject's general sense of well being [e.g., mental state, physical parameters, blood tests, electrocardiography (ECG or EKG), vital signs, mood, personal feelings, quality of life]. Recorder 150 can be, for example, a personal digital assistant (PDA), booklet of questionnaires (e.g., subject's symptom questionnaire diary), a hand held camera for photographing the skin reaction and optionally a computer software attached thereto, a transparent slide booklet for the subject to circle the reaction and a computer OMR system to scan and optionally input the results of the booklet questionnaires and/or the transparent slide booklet. Recorder 150 can be used by the treating physician (e.g., at the clinic) or the subject (e.g., who is located away from the clinic). The reaction on the skin, the level/state of clinical symptoms and/or the subject's general sense of well being which are recorded by recorder 150 can be inputted via user interface 140 to processing unit 120 for diagnosing the hypersensitivity, designing treatment regimen and/or monitoring the effect of treatment (treatment feedback) on the subject. Recorder 150 can be connected to [via connection 170 (which may be bi-directional), described below] user interface 140.

Connection 160, 170, 190 or 200 can be a wire [e.g., a universal serial bus (USB) cable, a network connection wire] or wireless (e.g., by radiofrequency, wireless network connection) connection. According to some embodiments of the invention, connection 170 can be via electronic mail (e-mail), to thereby send actual images of the reaction to the skin test to be analyzed or considered by the processing unit and optionally the treating physician.

According to some embodiments of the invention, the treatment regimen includes selecting hormone type, automated dosages of the hormones as calculated by the processing unit and timing of administration of desensitization treatment (e.g., by intradermal injections or reservoir patches). Such dosages can be calculated based on the data of the skin test results, the level of symptoms as recorded by the subject during treatment, additional subject's anamnesis data (e.g. BMI) and subject's general sense of well being and quality of life. In addition, the varying automated dosages can be administered using a syringe with retractable shaft (needle), or using a patch reservoir with predetermined doses.

The agents described hereinabove for diagnosing the medical condition associated with hypersensitivity of a subject to a female reproductive hormone (e.g., estriol, estradiol, estrone and/or progesterone) and optionally a lesion indicator for assessing a reaction to the female reproductive hormone on a skin may be included in a diagnostic kit/article of manufacture preferably along with appropriate instructions for use and labels indicating FDA approval for use in diagnosing a medical condition associated with hypersensitivity of a subject to a female reproductive hormone. The kit may also include appropriate buffers and preservatives for improving the shelf-life of the kit.

As used herein the phrase “lesion indicator” refers to the means for measuring the reaction to the female reproductive hormone on the skin of a subject. As described above, such means can be a camera, image analysis software and apparatus, a ruler, a transparent slide booklet which can be used for circling the reaction on the skin and optionally a computer OMR system to scan and optionally input the transparent slide booklet.

According to some embodiments of the invention, the lesion indicator enables determination of the presence of a reaction on the skin above the predetermined threshold as described above.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Development of a Skin Test for Premenstrual Syndrome

The present inventors have designed a new skin test for diagnosing premenstrual syndrome (PMS).

Study subjects—A female was considered “fertile” if having a regular menstrual cycle with regular ovulation. The normal length of the menstrual cycle in the reproductive age varies from 25 to 35 days and includes a luteal phase of 13-16 days. Most fertile females have a menstrual cycle of 27 to 31 days.

A female was considered “non-fertile” if being in menopause, having an irregular menstrual cycle, unovulatory menstrual cycle, oligo-ovulation, hypothyroidism, anorexia nervosa, polycystic ovarian syndrome (PCOS), and premature ovarian failure.

The skin test included the use of three fractions of estrogen (E1, E2 and E3) and progesterone (P), along with suitable controls. Table 1 depicts the hormone solutions which were used as antigens for the skin test. Each hormone solution was intradermally administered at a volume of 20-160 μl.

TABLE 1
The hormone solutions used in the skin test are provided
along with the dosages used (referred to as “A” hereinafter).
Hormone solutions

| Hormone | Dosage micrograms |
|---|---|
| 17 beta Estradiol Valerate (E2) | 7 |
| (or other estradiol) | |
| Estrone (E1) | 16 |
| Estriol (E3) | 17 |
| Progesterone (P) | 6 |

Table 2 depicts the solvents used in the skin test for diluting the hormones or the control solutions.

TABLE 2
Solvents which can be used for diluting the
hormones or as control solutions for the skin test.
Solvents used in the skin test
Solvents
Ethyl Oleate with 10% Benzyl Alcohol
Vegetable oil (such as sesame oil, peach oil)
Saline + 10% ethyl Alcohol
Present results were obtained using Ethyl Oleate, Sesame oil or Ethyl Alcohol as solvents.

Table 3 depicts the controls used in the skin test.

TABLE 3
The controls used in the skin test.
Controls used in the skin test
Controls used in the skin test
1% Histamine prick test [Histamine hydrochloride (10 mg/mL)
Solvent that is used in the skin test (any of the solvents in Table 2)
Saline [sodium chloride (9 grams/Liter)

Skin test method using estriol, estrone, estradiol and/or progesterone and monitoring the immediate, late and delayed immuno-reaction to the injected hormones

1. The hormones (Table 1) diluted in solvents (Table 2) and controls (Table 3) in the above described dosages were administered intradermally into subjects.

2. Timing of intradermal administration and evaluation of the skin reaction was dependent on the tested subject.

(i) In case of fertile woman: Intradermal administration of the hormones/controls initiated at the luteal phase of menstrual cycle;

Tracking (evaluation of skin reaction) was after 20 minutes, after 48 hours and daily during the next 30 day (or a complete menstrual cycle).

(ii) Other Cases (non-fertile subjects): Intradermal administration of the hormones/controls initiated at any time;

Tracking (evaluation of skin reaction) was after 20 minutes, after 48 hours and daily during the next 7 day.

3. Measurement of wheal diameter—The wheal is formed on the skin in response to the intradermal administration of the hormone. Since the wheal responses are rarely regular or circular the wheal diameter is calculated using the following formula I.

wheal diameter=(D+d)/2 Formula I:

where D is the maximum diameter and d is perpendicular diameter.

4. Comparison with the controls was as follows:

(i) In case of a negative reaction to histamine (wheal diameter smaller than 5 mm), the result indicates Familial Dysautonomia disease (the subject is advised to seek other treatment);

(ii) In case of a positive reaction to saline (wheal diameter equals or larger than 5 mm), the test should be repeated;

(iii) A result was considered positive if the wheal formed in response to the hormone exhibits a diameter which is more than 5 mm larger than that of the wheal formed in response to the solvent alone.

(iv) In case of a similar or smaller reaction (wheal diameter) as compared to solvent reaction, there is no sensitivity to the tested hormone and desensitization is optional;

(v) Exception—In case the skin reaction (wheal diameter) is larger than 20 mm, the subject should not be treated and is referred to testing of cancer, systemic auto-immune diseases and other serous diseases.

The diagnostic test described hereinabove is based on evaluation of a skin reaction to the tested hormones over an extended period of time, i.e., up to a complete menstrual cycle (28-30 days) in fertile women, or up to 7 days in non-fertile women. The late response, which is detected at these time periods, indicates an immuno-allergic reaction which involves cell-mediated immunological reaction (T-cells) to the female reproduction hormones (e.g., estrogens and progesterone). Detection of the late response (e.g., 7 days, 28-30 days), rather than the immediate or short-term response (e.g., after 20 minutes, 1 and 5 days described in Itsekson et al., 2004; and Govorukhina E. M. 1987) was found by the present inventors to be more accurate in diagnosing PMS.

### Example 2

### Development of PMS Treatment Regimen Based on the Novel Skin Test

Subjects exhibiting hypersensitivity to the tested hormones (as described in Examples 1 hereinabove) were treated according to the following method.

Evaluation of PMS symptoms—was according to the Visual Analogue Score (VAS) using a scale from 0 to 10 in which “0”=absence of symptoms, “1-3”=mild symptoms, “4-6”=moderate symptoms, “7-10” severe symptoms. The symptoms' evolution was assessed and described by each patient using a calendar of premenstrual experiences, which is a validated self-assessment daily diary (Thys-Jacobs S, Alvir J, Fratarcangelo P: Comparative analysis of three PMS assessment instruments: The identification of premenstrual syndrome with core symptoms. Psychopharmacol Bull 1995; 31:389-396). Subject selection of the patients with PMS met National Institute of Mental Health Premenstrual Syndrome Workshop criteria [Osofsky H J, Blumenthal S J (editors): Premenstrual Syndrome: Current Findings and Future Directions. Washington, American Psychiatric Press, 1985, p 88; Ekholm U B, Ekholm N O, Bäckström T. Premenstrual syndrome: comparison between different methods to diagnose cyclicity using daily symptom ratings; Acta Obstet Gynecol Scand. 1998, 77(5): 551-7; O'Brien P M, Abukhalil I E. Randomized controlled trial of the management of premenstrual syndrome and premenstrual mastalgia using luteal phase-only danazol. Am J Obstet. Gynecol. 1999, 180 (1 Pt 1):18-23].

Desensitization Methods

For desensitization subjects were administered intradermally according to the following treatment regimens:

Normal subjects (having a BMI of up to 25)

1. Treatment of fertile women—In case of treating a fertile woman, treatment was initiated during luteal phase of menstrual cycle according to the following regimens:

(a) In the first month, the dosage was A*2, i.e., twice the dosage of the hormone as indicated in Table 1 (Example 1 above), provided by a single intradermal administration per month.

(b) In the second month, the dosage was A*4, i.e., four times the dosage of the hormone as indicated in Table 1 (Example 1 above) provided by a single intradermal administration per month.

(c) In the third month, the dosage was A*8, i.e., eight times the dosage of the hormone as indicated in Table (Example 1 above), provided by a single intradermal administration per month.

2. Treatment of non-fertile women—In case of treating other subjects (i.e., non-fertile women), treatment was initiated at any time according to the following regimens:

(a) In the first week, the dosage was A*2, i.e., twice the dosage of the hormone as indicated in Table 1 above provided by a single intradermal administration per week.

(b) In the second week, the dosage was A*4, i.e., four times the dosage of the hormone as indicated in Table 1 (Example 1), provided by a single intradermal administration per week.

(c) In the third week, the dosage was A*8, i.e., eight times the dosage of the hormone as indicated in Table 1 (Example 1), provided by a single intradermal administration per week.

In case of BMI≧25 and reaction to Progesterone and not Estrogens—treatment regimens were as in a-c above (according to the fertility status of the subject).

In case of BMI≧25 and reaction to Estrogens and not Progesterone—treatment regimens were as in a-c above (according to the fertility status of the subject).

In case of BMI≧25 and reaction to both of Estrogens and Progesterone—treatment regimens included estrogen dosages which are larger than those of progesterone, as follows:

Administering estrogen
a. In the first month (in case of a fertile woman) or week (in case of a non-fertile woman), the dosage of estrogen was A*2, i.e., twice the dosage of the hormone as indicated in Table 1 (Example 1) provided by a single intradermal administration per month (in case of a fertile woman) or per week (in case of a non-fertile woman).
b. In the second month (in case of a fertile woman) or week (in case of a non-fertile woman), the dosage of estrogen was A*4, i.e., four times the dosage of the hormone as indicated in Table 1 (Example 1), provided by a single intradermal administration per month (in case of a fertile woman) or per week (in case of a non-fertile woman).
c. In the third month (in case of a fertile woman) or week (in case of a non-fertile woman), the dosage of estrogen was A*8, i.e., eight times the dosage of the hormone as indicated in Table 1 (Example 1), provided by a single intradermal administration per month (in case of a fertile woman) or per week (in case of a non-fertile woman).

Administering progesterone
a. In the first month (in case of a fertile woman) or week (in case of a non-fertile woman), the dosage of progesterone was A, i.e., the dosage of the hormone as indicated in Table 1 (Example 1), provided by a single intradermal administration per month (in case of a fertile woman) or per week (in case of a non-fertile woman).
b. In the second month (in case of a fertile woman) or week (in case of a non-fertile woman), the dosage of progesterone was A*2, i.e., twice the dosage of the hormone as indicated in Table 1 (Example 1), provided by a single administration per month (in case of a fertile woman) or per week (in case of a non-fertile woman).
c. In the third month (in case of a fertile woman) or week (in case of a non-fertile woman), the dosage of estrogen was A*4, i.e., four times the dosage of the hormone as indicated in Table 1 (Example 1), provided by a single intradermal administration per month (in case of a fertile woman) or per week (in case of a non-fertile woman).

Desensitization treatment: ratio between hormones in each injection—In addition, the ratio between estrogen and progesterone in the intradermal injections was selected according to the body mass index (BMI) of the treated subject, as follows.
1. In case BMI=25 (BMI equals 25), the ratio between estrogen and progesterone in each injection was 1:1.
2. In case 25<BMI<29 (BMI higher than 25 and lower than 29), the ratio between estrogen and progesterone in each injection was 2:1.
3. In case 29<BMI<35 (BMI is higher than 29 but lower than 35), the ratio between estrogen and progesterone in each injection is 3:1.
4. In case of morbidity and BMI>35 (BMI higher than 35), the ratio between estrogen and progesterone in each injection is 1:1 and additional factors such as diabetes and hypertension are considered.

Treatment feedback—For best results, after desensitization with the hormone, skin reaction and symptom severity were tracked daily. Before administering the next set of injections, an assessment of skin reaction and PMS symptoms [evaluated according to the visual analogue scores (VAS) from 0 to 10 of the dominant symptoms] was performed using the following decision rules (Table 2).

TABLE 4
Criteria used in feedback control of treatment.
Treatment feedback

| Skin Test | Symptoms | Treatment Result |
|---|---|---|
| − | − | Continue treatment as planned |
| 0 | 0 | Continue treatment as planned |
| − | + | Stop and continue to other treatments/tests |
| + | − | Continue treatment as planned |
| + | + | Stop and continue to other treatments/tests |
| 0 | − | Continue treatment as planned |
| − | 0 | Add additional cycle same as last-but no more than 4 cycles in total |

Skin test results include: Decrease in skin test reaction (−); increase in skin test reaction (+); no change in skin test reaction (0).
PMS symptoms were evaluated based on the VAS criteria described above and included: decrease (−); increase (+); or no change (0) in PMS symptoms after treatment.
The overall severity is a combination of number of days and severity level (severe symptoms = VAS scores from 7-10 on the scale).
The overall sensitivity is a combination of weal and flare size and days of its appearance.

Magnesium depletion—Menstrual related pain, cognitive, emotional and other conditions (e.g. PMS and Menopause) are usually accompanied by intracellular magnesium fluctuation and sometimes depletion of magnesium [Li W, Zheng T, Altura B M, Altura B T. Brain Res Bull. 2001 Jan. 1; 54(1):83-9]. Therefore, treatment that addresses such medical conditions is usually accompanied by supplemental magnesium which normalizes intracellular magnesium in patients.

The present inventors have uncovered that the desensitization treatment can be supplemented with magnesium at a dosage of 10-30 mg/kg/day of magnesium applied to the reaction place at hyper sensitivity timing. Non-limiting examples of magnesium supplements which can be used to treat the symptoms of the medical condition include magnesium tablets (e.g., “Anti Leg Cramps” by Nave Pharm, Israel, which also includes—10 mg Vitamin B and 50 mg Vitamin E); a magnesium patch; a magnesium cream or a magnesium gel. The inventors have uncovered that magnesium should be given at the hypersensitivity timing along with the desensitization therapy. In addition, if the desensitization is not feasible (e.g. the patient dis-consent), the treatment with magnesium should be given only at hypersensitivity timing.

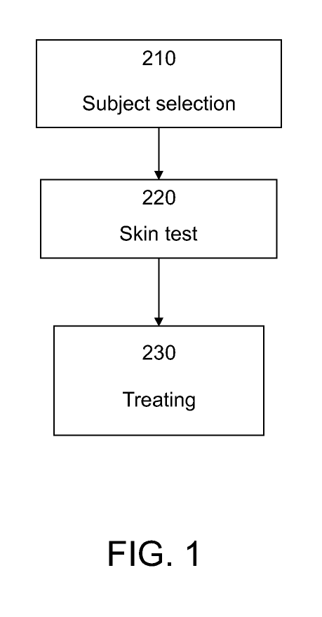

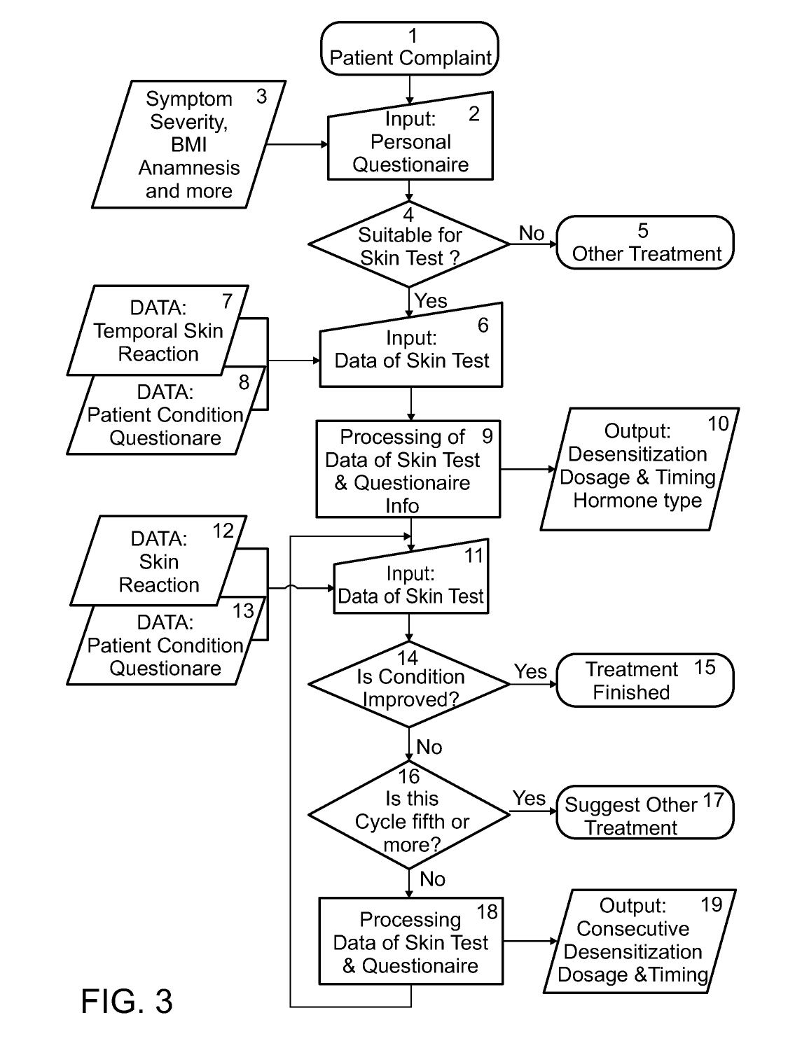

FIGS. 1 and 3 schematically illustrate algorithms for diagnosing and optionally treating a medical condition associated with hypersensitivity to a female reproductive hormone according to some embodiments of the invention.

### Example 3

### Validation of the Novel Method of Treating PMS

Study subjects—Total of 73 patients with PMS were included in the study. The ages of the PMS subjects varied from 18-55 years old (see Table 5 below for subjects' characteristics). The study was performed under approval by Helsinki committee.

Study design—Comparative, longitudinal (before-after), two-arms, observational, non-randomized, non-blind study. Each patient chose either a standard treatment (group 0) or a new treatment (group 1). The standard treatment included Regular Mg supplement and B6 vitamin supplement.

Data—For each patient, data regarding age, number of children, and body mass index (BMI) were recorded. Each patient filled a questionnaire with 19 questions at the beginning of the study and after 6 month of treatment. The answers of each question were integer numbers from 0 to 10, meaning the absence of a symptom (O) or extremely high symptom (10). The overall level of PMS was estimated as mean value of all 19 answers (symptoms).

Statistical methods—Since the study was not randomized, the first step was the comparison of the initial status of the two groups (Table 5, below). The comparisons were done twice, using Student non-paired t-test, and Mann-Whitney rank sum test. The latter is preferable because the scale is not interval, rather an ordinal. However, t-test was used for comparison with similar studies. Two possible outcomes were considered. One is the symptom level at the end of treatment (i.e., by 6 months) and the second is the difference between the levels of a symptom at the beginning of study and after 6 months. Because the final level strongly depends on the initial one, the most appropriate method used was ordinal logistic regression (appropriate for ordinal scale). However, the initial level of a symptom was considered as interval measured because of limited sample size. The results of comparisons of the final level by Mann-Whitney, t-test, and ordinal logistic regression are shown in Table 6 below.

Additional outcome was the difference between last and first level of a symptom. It was also compared by Mann-Whitney and Student's t-tests (Table 7). Since each of the 19 symptoms were tested (a multiple comparison), the Benjamini-Hochberg FDR procedure was used for testing significance of the differences between groups after adjustment for 19 comparisons. The results, that stayed significant after this correction are marked by *.

The initial and final overall levels of PMS were compared between groups using t-test and Mann-Whitney rank sum test. The absolute improvement was also analyzed by linear regression of the final level on the group, adjusted by the initial level. The relative effect on the overall level of PMS was analyzed in log-scale.

All p-values are two sided. The data are presented as mean+/−SD.
All calculations were performed using STATA 8SE software.

Statistical Results and analysis—Two group comparisons.

TABLE 5
General characteristics of the data including both treatment groups

| | No. of | | | | |
|---|---|---|---|---|---|
| Variable | subjects | Mean | Std. Dev. | Minimum | Maximum |
| Age | 73 | 36.09589 | 9.755574 | 18 | 55 |
| Body mass | 73 | 23.36545 | 3.617834 | 18.82711 | 34.72222 |
| index (BMI) | | | | | |
| Before | | | | | |
| Depression | 73 | 6.178082 | 3.35552 | 0 | 10 |
| Mood change | 73 | 6.041096 | 3.177343 | 0 | 10 |
| Stress | 73 | 5.356164 | 3.683182 | 0 | 10 |
| Anxiety | 73 | 5.054795 | 3.733818 | 0 | 10 |
| Angry | 73 | 5.315068 | 3.829261 | 0 | 10 |
| Crying | 73 | 3.917808 | 3.569755 | 0 | 10 |
| Leg swell_b | 73 | 3.150685 | 3.611983 | 0 | 10 |
| Mastalgia | 73 | 5.534247 | 3.70466 | 0 | 10 |
| Bloating | 73 | 6.041096 | 3.549015 | 0 | 10 |
| Cramps | 73 | 5 | 3.700601 | 0 | 10 |
| General pain | 73 | 4.986301 | 3.717426 | 0 | 10 |
| Low back pain | 73 | 6.191781 | 3.67294 | 0 | 10 |
| Headache | 73 | 6.39726 | 3.736976 | 0 | 10 |
| Tiredness | 73 | 6.863014 | 3.23314 | 0 | 10 |
| Appetite shifts | 73 | 5.575342 | 3.858316 | 0 | 10 |
| Crave for sweets | 73 | 5.547945 | 3.961766 | 0 | 10 |
| Sleeping disorder | 73 | 4.438356 | 3.975572 | 0 | 10 |
| Acne | 73 | 3.890411 | 8.160817 | 0 | 66 |
| Itch ef | 73 | 2.808219 | 3.561591 | 0 | 10 |
| After | | | | | |
| Depression | 73 | 3.835616 | 3.184222 | 0 | 10 |
| Mood change | 73 | 3.972603 | 3.144539 | 0 | 10 |
| Stress | 73 | 3.534247 | 3.158244 | 0 | 10 |
| Anxiety | 73 | 3.369863 | 3.229667 | 0 | 10 |
| Angry | 73 | 3.657534 | 3.355009 | 0 | 10 |
| Crying | 73 | 2.315068 | 2.8474 | 0 | 10 |
| Leg swell_b | 73 | 2.054795 | 2.753232 | 0 | 10 |
| Mastalgia | 73 | 3.273973 | 3.375814 | 0 | 10 |
| Bloating | 73 | 4.082192 | 3.165585 | 0 | 10 |
| Cramps | 73 | 3.452055 | 3.407676 | 0 | 10 |
| General pain | 73 | 3.09589 | 3.064874 | 0 | 10 |
| Low back pain | 73 | 3.876712 | 3.278487 | 0 | 10 |
| Headache | 73 | 3.972603 | 3.261614 | 0 | 10 |
| Tiredness | 73 | 4.890411 | 3.195316 | 0 | 10 |
| Appetite shifts | 73 | 4.739726 | 3.427938 | 0 | 10 |
| Crave for sweets | 73 | 3.630137 | 3.272388 | 0 | 10 |
| Sleeping disorder | 73 | 3.013699 | 3.255308 | 0 | 10 |
| Acne | 73 | 2.109589 | 2.811493 | 0 | 10 |
| Itch ef | 73 | 1.438356 | 2.327292 | 0 | 10 |
| Improvement | | | | | |
| Depression | 73 | 2.342466 | 3.096679 | 4 | 10 |
| Mood change | 73 | 2.068493 | 2.820209 | 9 | 8 |
| Stress | 73 | 1.821918 | 2.931356 | 6 | 10 |
| Anxiety | 73 | 1.684932 | 2.871685 | 7 | 10 |
| Angry | 73 | 1.657534 | 2.982445 | 8 | 10 |
| Crying | 73 | 1.60274 | 2.832057 | 3 | 8 |
| Leg swell_b | 73 | 1.09589 | 3.127674 | 10 | 9 |
| Mastalgia | 73 | 2.260274 | 3.261848 | 4 | 10 |
| Bloating | 73 | 1.958904 | 2.820748 | 5 | 10 |
| Cramps | 73 | 1.547945 | 2.910904 | 6 | 10 |
| General pain | 73 | 1.890411 | 2.903837 | 2 | 10 |
| Low back pain | 73 | 2.315068 | 3.077141 | 2 | 10 |
| Headache | 73 | 2.424658 | 3.303692 | 7 | 10 |
| Tiredness | 73 | 1.972603 | 2.748599 | 3 | 9 |
| Appetite shifts | 73 | .8356164 | 2.981488 | 9 | 10 |
| Crave for sweets | 73 | 1.917808 | 3.130289 | 6 | 10 |
| Sleeping disorder | 73 | 1.424658 | 2.948247 | 5 | 10 |
| Acne | 73 | 1.780822 | 7.676165 | 5 | 64 |
| Itch ef | 73 | 1.369863 | 3.146898 | 8 | 10 |

TABLE 6
Comparisons of the groups: Initial status

| | Group 0 (Others) | | Group1 | | Mann | |
|---|---|---|---|---|---|---|
| Variable | Mean ± SD | | Mean ± SD | | Whitney | T test |
| Age | 35.44444 | 10.01665 | 36.47826 | 9.690176 | | 0.6651 |
| Number of | 1.259259 | 1.318291 | 1.195652 | 1.720718 | | 0.8690 |
| children | | | | | | |
| Body mass | 23.49428 | 3.908846 | 23.28983 | 3.478254 | | 0.8175 |
| index | | | | | | |
| Depression | 5.592593 | 3.805454 | 6.521739 | 3.053152 | 0.4527 | 0.2562 |
| Mood change | 5.777778 | 3.202563 | 6.195652 | 3.187612 | 0.4259 | 0.5910 |
| Stress | 4.962963 | 3.705313 | 5.586957 | 3.691288 | 0.4886 | 0.4885 |
| Anxiety | 4.444444 | 3.826359 | 5.413043 | 3.673183 | 0.3119 | 0.2877 |
| Angry | 5.111111 | 4.181768 | 5.434783 | 3.649366 | 0.9445 | 0.7300 |
| Crying | 4 | 3.812933 | 3.869565 | 3.46159 | 0.9159 | 0.8814 |
| Leg swell_b | 2.666667 | 3.562627 | 3.434783 | 3.649366 | 0.3663 | 0.3841 |
| Mastalgia | 5.185185 | 3.922686 | 5.73913 | 3.59898 | 0.5860 | 0.5411 |
| Bloating | 6.333333 | 3.86304 | 5.869565 | 3.383678 | 0.3740 | 0.5934 |
| Cramps | 5.074074 | 3.740896 | 4.956522 | 3.717565 | 0.9216 | 0.8968 |
| General pain | 3.703704 | 3.677529 | 5.73913 | 3.567973 | 0.0313 | 0.0228 |
| Low back pain | 5.407407 | 3.629571 | 6.652174 | 3.658885 | 0.0691 | 0.1637 |
| Headache | 4.962963 | 3.847891 | 7.23913 | 3.43954 | 0.0043 | 0.0110 |
| Tiredness | 6.333333 | 3.430631 | 7.173913 | 3.107728 | 0.2614 | 0.2867 |
| Appetite shifts | 4.888889 | 4.116882 | 5.978261 | 3.684738 | 0.3164 | 0.2469 |
| Crave for | 4.518519 | 4.126559 | 6.152174 | 3.776932 | 0.0605 | 0.0891 |
| sweets | | | | | | |
| Sleeping | 3.296296 | 3.969255 | 5.108696 | 3.86568 | 0.0665 | 0.0595 |
| disorder | | | | | | |
| Acne | 2.925926 | 3.781798 | 4.456522 | 9.869609 | 0.5183 | 0.4430 |
| Itch ef | 2.222222 | 3.273828 | 3.152174 | 3.711648 | 0.3142 | 0.2846 |

Table 6: Shown are the values of the tested variables at study initiation (day zero) in Group 0 (patients treated by standard PMS therapies) and Group I (patients treated according to the new treatment regimens as described in Example 2 hereinabove).

TABLE 7
Comparisons of the group: Final status

| | Group 0 (Others) | | Group1 | | Mann | | Ordinal |
|---|---|---|---|---|---|---|---|
| Variable | Mean ± SD | | Mean ± SD | | Whitney | T test | LR |
| Depression | 5.07407 | 3.63662 | 3.10869 | 2.66856 | 0.0244 | 0.0099* | 0.000* |
| Mood change | 5.55555 | 3.35505 | 3.04347 | 2.63275 | 0.0024* | 0.0007* | 0.000* |
| Stress | 4.88888 | 3.59843 | 2.73913 | 2.59438 | 0.0173 | 0.0042* | 0.000* |
| Anxiety | 4.22222 | 3.88622 | 2.86956 | 2.69657 | 0.1857 | 0.0841 | 0.001* |
| Angry | 5 | 3.98072 | 2.86956 | 2.67173 | 0.0307 | 0.0079* | 0.000* |
| Crying | 3.22222 | 3.72448 | 1.78260 | 2.04301 | 0.3193 | 0.0361 | 0.082 |
| Leg swell_b | 2.14814 | 3.37073 | 2 | 2.35702 | 0.3903 | 0.8261 | 0.875 |
| Mastalgia | 5.07407 | 4.18721 | 2.21739 | 2.24016 | 0.0115 | 0.0003* | 0.000* |
| Bloating | 5.11111 | 3.87629 | 3.47826 | 2.51872 | 0.0674 | 0.0324 | 0.011* |
| Cramps | 4.59259 | 3.91505 | 2.78260 | 2.91290 | 0.0631 | 0.0274 | 0.003* |
| General pain | 2.96296 | 3.56782 | 3.17391 | 2.76730 | 0.4638 | 0.7787 | 0.099 |
| Low back pain | 4.25925 | 3.77840 | 3.65217 | 2.96810 | 0.5042 | 0.4488 | 0.028 |
| Headache | 4.25925 | 3.83899 | 3.80434 | 2.90302 | 0.5823 | 0.5687 | 0.004* |
| Tiredness | 6.18518 | 3.46451 | 4.13043 | 2.79371 | 0.0051* | 0.0071* | 0.000* |
| Appetite shifts | 5.18518 | 4.06710 | 4.47826 | 3.00916 | 0.3392 | 0.3987 | 0.019* |
| Crave for sweets | 3.18518 | 3.90302 | 3.89130 | 2.85367 | 0.1496 | 0.3771 | 0.903 |
| Sleeping disorder | 3.11111 | 3.83639 | 2.95652 | 2.9056 | 0.7713 | 0.8463 | 0.063 |
| Acne | 2.40740 | 3.47805 | 1.93478 | 2.36081 | 0.9511 | 0.4919 | 0.581 |
| Itch ef | 1.70370 | 2.87983 | 1.28260 | 1.95121 | 0.9535 | 0.4593 | 0.213 |

Table 7: Shown are the values of the tested variables at end of study (6 months after beginning of treatment) in Group 0 (patients treated by standard PMS treatments) and Group I (patients treated according to the new treatment regimens as described in Example 2 hereinabove).

TABLE 8
Comparisons of the group: Improvement over 6 months

| | Group 0 (Others) | | Group1 | | Mann | |
|---|---|---|---|---|---|---|
| Variable | Mean ± SD | | Mean ± SD | | Whitney | T test |
| Depression | .5185185 | 2.547553 | 3.413043 | 2.902689 | 0.0000* | 0.0001 |
| Mood change | .2222222 | 2.391223 | 3.152174 | 2.485222 | 0.0000* | 0.0000 |
| Stress | .0740741 | 1.685365 | 2.847826 | 3.032911 | 0.0000* | 0.0000 |
| Anxiety | .2222222 | 1.717183 | 2.543478 | 3.074674 | 0.0000* | 0.0006 |
| Angry | .1111111 | 2.708013 | 2.565217 | 2.778106 | 0.0000* | 0.0005 |
| Crying | .7777778 | 2.650593 | 2.086957 | 2.850544 | 0.0212* | 0.0560 |
| Leg swell_b | .5185185 | 3.389694 | 1.434783 | 2.948839 | 0.2282 | 0.2294 |
| Mastalgia | .1111111 | 1.717183 | 3.521739 | 3.304805 | 0.0013* | 0.0000 |
| Bloating | 1.222222 | 3.29724 | 2.391304 | 2.435645 | 0.0000* | 0.0874 |
| Cramps | .4814815 | 2.063797 | 2.173913 | 3.164416 | 0.0091* | 0.0154 |
| General pain | .7407407 | 2.030396 | 2.565217 | 3.138663 | 0.0013* | 0.0086 |
| Low back pain | 1.148148 | 2.460514 | 3 | 3.218005 | 0.0037* | 0.0120 |
| Headache | .7037037 | 2.997625 | 3.434783 | 3.074282 | 0.0022* | 0.0004 |
| Tiredness | .1481481 | 1.321529 | 3.043478 | 2.812326 | 0.0000* | 0.0000 |
| Appetite shifts | .2962963 | 2.613792 | 1.5 | 3.009245 | 0.0045* | 0.0119 |
| Crave for sweets | 1.333333 | 3.2463 | 2.26087 | 3.043644 | 0.0091* | 0.2241 |
| Sleeping disorder | .1851852 | 2.131035 | 2.152174 | 3.133811 | 0.0014* | 0.0051 |
| Acne | .5185185 | 1.503083 | 2.521739 | 9.563215 | 0.0374* | 0.2848 |
| Itch ef | .5185185 | 2.137043 | 1.869565 | 3.537788 | 0.0091* | 0.0764 |

Table 8: Shown are the improvements over the period of study (6 months) calculated by subtracting the values at the end of study from those at the beginning of study (day zero) in both Group 0 (patients treated by standard PMS therapies) and Group I (patients treated according to the new treatment regimens as described in Example 2 hereinabove).

Overall effect—Assuming that the scale is interval and all symptoms have the same importance, the overall level of PMS can be measured using mean score of all 19 symptoms. The overall level of PMS before the study in the two groups was 4.60039±0.4223931 in group 0 (other treatments) and 5.509153±0.324051 in group 1 (desensitization treatment). Thus, the PMS symptoms in group 1 was a little stronger, but the difference was not significant (t-test: P-value=0.0923, Mann-Whitney: P-value=0.1374). After 6 months the numbers were 4.11306±2.246085 in group 0, and 2.957666±1.553464 in group 1. This difference was significant (t-test: P-value=0.0115, Mann-Whitney: P-value=0.0340). The improvement, measured as the difference between the levels after 6 month and the initial level, was 0.4873294±1.461604 in group 0, and 2.551487±1.686429 in group 1. It was stronger in the Desensitization group (Group 1) than in the other treatment group (Group 0; t-test: p-value<0.0001, Mann-Whitney:P-value<0.0001). The relative improvement in log scale was 0.2163505±0.6179286 in group 0, and 0.7440191±0.6267285 in group 1. The difference in the effect was highly significant (t-test: P-value=0.0008, Mann-Whitney: P-value<0.0001).

The regression of the final level adjusted for initial level showed that the final level in group 1 was lower by 1.68436±0.3288011 in the same initial level of PMS. This difference was highly significant (P<0.0001). The relative improvement was also stronger in group 1 (log of effect=0.5592043±0.1537736, with p-value=0.0001). It means that the final level in group 1 was almost two times lower than in group 0 (0.57%) at the same initial level.

Summary—In spite of the fact that the study was not randomized, the groups were comparable in most symptoms, except 3. The effect of desensitization was stronger in almost all symptoms, as compared by final status, or by improvement in separate symptoms. After adjustment for initial level the differences became even stronger. The overall level of PMS, measured by mean value of all 19 symptoms did not differ significantly at the initial point, but was significantly lower in desensitization group after 6 month. The absolute improvement in the desensitization group (group 1) was 2.55 versus 0.56 in other treatment group (group 0; P<0.0001). The relative improvement was 47% in desensitization group versus 19% in other group (P<0.0001).

### Example 4

### Skin Test as a Feedback for Other Treatments

Skin Test as a Feedback for Other Treatments

The present inventors have uncovered that the diagnostic skin test described in Example 1, hereinabove, can be utilized for improving the therapeutic effect of PMS or similar syndromes by standard treatments. In many cases, anti-hormonal therapy such as using the anti-estrogen agent Tamoxifen 10 (GENERICS LTD, ENGLAND) or the anti-progesterone agent Mifepristone, known as RU-486 (Roussel Uclaf), is associated with side effects. Using the skin test described hereinabove, the anti-hormonal therapy can be restricted to the time period (e.g., certain days throughout the menstrual cycle) in which the subject exhibits hypersensitivity to the hormone. This is in contrast to administering the drug on a regular daily schedule. In addition, treatment of symptoms such as headaches which are associated with the menstrual cycle can be restricted to the days in which the subject exhibits hypersensitivity to hormones. Thus, the diagnostic method can be used for a timely-coordinated individual therapy, adjusted according to the level of hypersensitivity to hormones as measured in the subject.

### Example 5

### Diagnosis and Treating PMS and Infertility

Following is a description of several cases of subjects which were diagnosed and treated according to the method of some embodiments of the invention. The concentrations of hormones used in the description of the following cases 1-7 and in Table 9 below were as follow:

E1 (Estrone)—3 mM (millimole/Liter); 0.811 grams/liter;

E2 —(estradiol)—1 mM (millimole/Liter); 0.3565 grams/liter;

E3 (estriol)—3 mM (millimole/Liter); 0.86514 grams/liter;

P (progesterone)—1 mM (millimole/Liter); 0.3144 grams/liter;

Case 1:

A-33-year-old female, born in weight of 2200 grams. Presently, BMI=31 (overweight). Regular menstruation started at age 18. She has complained on strong premenstrual headaches (VAS-10) 3 days up to menstrual flow (which results in loss of 3 work days, monthly); insomnia (VAS-8), and acne (VAS-9).

At the age of 26—the first spontaneous pregnancy. During pregnancy since the first days she has suffered from sever hyperemesis gravidarum with loss of weight (8 kg) and repeatedly needed hospitalization for intra venous (I.V.) fluid administration. Spontaneous delivery in 36 weeks of gestation, newborn female with a birth weight of 2150 grams.

After the first pregnancy, the premenstrual headaches have continued and worsened (duration from 3 days to 5 days and the rank of the pain was increased).

The diagnosis: Severe premenstrual headaches, insomnia, premenstrual acne and obesity.

Skin test results: delayed-type hypersensitivity reaction to estriol and estradiol, higher than to Progesterone.

Desensitization Treatment According to the Computing Platform of the Invention:

### Month 1: 20 μl Estriol, 20 μl Estradiol, 10 μl Progesterone;

### Month 2: 40 μl Estriol, 40 μl Estradiol, 20 μl Progesterone;

### Month 3: 80 μl Estriol, 80 μl Estradiol, 40 μl Progesterone;

Following 3 months of desensitization treatment no hypersensitivity to the female reproductive hormone was observed. Questionnaire shows significant decrease in symptoms severity.

Case 2

A 30-year-old woman with a 4-year history of infertility (4 Consecutive recurrent habitual abortions before 12 weeks) presented with irregular menstruation cycles with 21- to 45 days intervals. BMI=24.6. Except for infertility, complaints of heavy headaches and pains in breasts (Mastalgia) in premenstrual periods (VAS 10).

Treatment of PMS by oral contraceptives (YAS) has resulted in amplification of headaches and Mastalgia.

The diagnosis: Habitual abortions and severe PMS.

Skin test results: A positive delayed-type hypersensitivity reaction to estrone, estradiol and progesterone.

Desensitization treatment according to the computing platform of the invention:

### Month 1: 40 μl Estrone, 40 μl Estradiol, 40 μl Progesterone;

### Month 2: 80 μl Estrone, 80 μl Estradiol, 80 μl Progesterone;

### Month 3: 160 μl Estrone, 160111 Estradiol, 160 μl Progesterone;

After the desensitization treatment to estrone, estradiol and progesterone, the menstrual cycle became regular 28-30 days. Premenstrual headaches and pains in breasts have decreased up to mild PMS (VAS-4)

In 2 months after the ending of treatment has spontaneously become pregnant. During the pregnancy, the fetus and the pregnant female were in good condition. At 39 weeks-of gestation, the patient had delivered a normal 3750 gram newborn male.

After delivery there were no complaints of PMS.

After 2 years repeated spontaneous pregnancy and normal delivery in 39 weeks of gestation of a normal newborn female 3550 gram.

Case 3:

A-31-year-old female with a 3 year history of infertility (3 consecutive recurrent habitual abortions before 12 weeks) presented with regular menstruation cycles with 28 day intervals. BMI=24.2.

Except for infertility there are complaints of depression (treatment with Prosac), spastic bronchitis, atopic dermatitis, all accompanied by severe PMS. Treatment by oral contraceptives resulted in worsening of depression syndromes.

The diagnosis: Habitual abortions; and severe PMS.

Skin test results: Skin tests revealed delayed-type hypersensitivity reaction to estrone and estradiol.

Desensitization Treatment According to the Computing Platform of the Invention:

### Month 1: 40 μl Estrone, 40 μl Estradiol,

### Month 2: 80 μl Estrone, 80 μl Estradiol,

### Month 3: 160 μl Estrone, 160 μl Estradiol,

The results of the desensitization treatment were abolishment of PMS and atopic dermatitis symptoms. Prozac treatment was stopped. Weight loss of 7 kg.

In addition, following desensitization treatment there was a spontaneous pregnancy and uncomplicated delivery at 40 weeks of gestation to a healthy male newborn of 3440 grams. Absence of PMS and atopic dermatitis symptoms after the pregnancy.

Case 4:

A-40-year-old female with a 15 years history of infertility (11 consecutive recurrent habitual abortions before 12 weeks), presented with regular menstruation cycles with 30-31 day intervals. BMI=23.4.

Except for infertility, complaints to severe PMS (VAS 10) which included depression, heavy headaches, low back pains, mastalgia and dysmenorrhea.

Previous treatments include 6 cycles of Intra-Utero Insemination (IUI) and 8 cycles of in vitro fertilization (IVF) and a combination with immunoglobulin. Treatments were unsuccessful during 5 years.

The Diagnosis: Recurrent habitual abortions; and severe PMS.

Skin test results: Skin tests revealed delayed-type hypersensitivity reaction to estrone, estriol, estradiol and progesterone.

Desensitization Treatment According to the Computing Platform of the Invention:

### Month 1: 40 μl Estrone, 40 μl Estradiol, 40 μl Estriol, 40 μl Progesterone;

### Month 2: 80 μl Estrone, 80 μl Estradiol, 80 μl Estriol, 80 μl Progesterone;

### Month 3: 160 μl Estrone, 160 μl Estradiol, 160 μl Estriol, 160 μl Progesterone;

After the desensitization treatment the PMS symptoms declined and diagnosis has changed to mild PMS (VAS-4).

After additional treatment by two cycles of IVF and Pre-gestational Genetic Diagnosis (PGD), pregnancy was successful.

At the 24^{th} week of gestation the fetus was in good condition. At 38 weeks of gestation, the patient has delivered a healthy newborn female of 2385 grams. Neonatal course was uneventful.

Case 5

Female, 48 years of age, married+1 child, complaints of hot flashes up 20 flashes/day, night sweats which result in insomnia, chronic fatigue, tendency to cry, irritability, vertigo for the last 10 months—which coincided with change in the menstrual cycle. Only 3 menstrual flows (of up to 10-12 days) took place in this period.

Normal cycle (every 25 days) since the age of 13, head aches, mastalgia and abdominal pains before and in the first day of menstruation.

Three pregnancies, of them one medical abortion, one normal pregnancy and labor (girl of 3100 g) and one extrauterine pregnancy (in the left tube). Does not suffer from chronic diseases, does not use medications (including hormonal). Laboratory tests: FSH=35 mIU/ml; LH=24 mIU/ml.

The diagnosis: Premenopausal syndrome, hot flashes (severe), night sweats (severe), insomnia, fatigue syndrome.

Skin test results: Skin tests revealed delayed-type hypersensitivity reaction (after 48 hours) to estriol, estrone and progesterone.

Desensitization Treatment According to the Computing Platform of the Invention:

### Week 1: 40 μl Estrone, 40 μl Estriol, 40 μl Progesterone;

### Week 2: 80 μl Estrone, 80 μl Estriol, 80 μl Progesterone;

### Week 3: 160 μl Estrone, 160 μl Estriol, 160 μl Progesterone;

After the desensitization treatment all the “severe” diagnosis was reduced to “mild”; and insomnia and fatigue were absent. The positive clinical effect maintained for the one year follow up.

According to patient's request (increased stress which was associated with deterioration of the medical condition), the desensitization the course was repeated twice in 4 years, with marked clinical improvement, and good quality of life during last two years menopause (no hot flashes and night sweats).

Case 6

A female of 50 years old (one child), complained on hot flashes up to 15 flashes/day mostly at nights, bloating, insomnia, general weakness. Complaints are of 6 months, attributes her condition to a menopause, which began one year earlier.

Regular menstrual cycle since 12 years of age with common premenstrual pains and bloating.

Single pregnancy and labor at age of 36 (healthy boy of 2800 grams). The pregnancy was accompanied by nausea and vomiting, loss of weight and repeated hospitalizations. Bloating and pre-menstrual pains increased post labor. Smokes 10 cigarettes/day.

Hormonal therapy (Progyluton) was stopped due to side effects (nausea and Mastalgia).

BMI=23.8. Laboratory tests: FSH=33 mIU/ml, LH=21 mU/ml.

The diagnosis: Menopausal syndrome, characterized by hot flashes (severe), insomnia, bloating, and fatigue.

Diagnostic skin test: Hypersensitivity (delayed type after 48 hours and 5 days) to estriol, estrone and estradiol.

Desensitization Treatment According to the Computing Platform of the Invention:

### Week 1: 40 μl Estrone, 40 μl Estriol, 40 μl Estradiol;

### Week 2: 80 μl Estrone, 80 μl Estriol 80 μl Estradiol;

### Week 3:160 μl Estrone, 160 μl Estriol 160 μl Estradiol;

Desensitization treatment resulted in significant clinical improvement. The severe hot flashes were reduced to mild. Insomnia and fatigue are absent. Positive clinical effect remained in the two years follow up. Good quality of life (no hot flashes).

Case 7:

A 53-years-old female, BMI=23.8; 5 pregnancies, of them 3 labors (two normal pregnancy and delivery and one premature labor at 32 weeks complicated by deep vein thrombosis) and two spontaneous abortions.

Complaints for the last 3 years (attributes her condition to the menopause at age 48) include: heart palpitations, hot flashes up to 12-20 episodes/day (especially at nights), painful urination, insomnia, general weakness, acne, bloating,

Menstruated since 12 years of age, regularly every 28 days, repeatedly suffered acne, irritability and back-pains prior to menstruations. Pre menstrual symptoms were ameliorated after labors.

Used oral contraceptives for many years—with no side effects.

Currently under medical attention and receives care for recurrent vaginal herpes, chronic urinary tract infection (UTI) and suspected interstitial cystitis. Uses Minocin and Sedural for acne and UTI. Cardiological and endocrinological pathologies were excluded by specialists.

Laboratory tests: LH=80 IU/l, FSH=136 IU/l.

The diagnosis: Menopause, hot flashes (severe), chronic urinary tract infection (UTI), suspected interstitial cystitis.

Skin test results: Skin tests revealed delayed-type (after 48 hours and 6 days) hypersensitivity reaction to estrone, estradiol and progesterone.

Desensitization Treatment According to the Computing Platform of the Invention:

### Week 1: 40 μl Estrone, 40 μl Estradiol, 40 μl Progesterone;

### Week 2: 80 μl Estrone, 80 μl Estradiol 80 μl Progesterone;

### Week 3: 160 μl Estrone, 160 μl Estradiol 160 Progesterone.

Desensitization treatment resulted in lessening of hot flashes and heart palpitations, normal sleep had been restored.

Follow up examination after 7 months revealed no worsening in the condition.

Table 9, below summarizes the diagnosis and treatment of various medical conditions associated with hypersensitivity to a female reproductive hormone according to the teachings of the invention.

TABLE 9
Diagnosis and treating of medical conditions associated with hypersensitivity to a
female reproductive hormone according to some embodiments of the invention.

| | | | | | State of medical |
|---|---|---|---|---|---|
| | | | | | condition following |
| Case | Age | | Additional | | desensitization |
| No. | (Years) | Main diagnosis | diagnosis | Skin test results | treatment |
| 1 | 33 | PMS syndrome | During | Hypersensitivity to | Significant decrease |
| | | (VAS-10) | pregnancy: | estriol and estradiol, | in symptom severity |
| | | | Hyperemesis | which is higher than | |
| | | | gravidarium | to progesterone | |
| 2 | 30 | Infertility, recurrent | Severe PMS | Hypersensitivity to | Regular menstrual |
| | | habitual abortions | syndrome | estrone, estradiol | cycle; mild PMS |
| | | | (VAS-10) | and progesterone. | (VAS = 4); |
| | | | | | Two spontaneous |
| | | | | | pregnancies with |
| | | | | | normal deliveries. |
| 3 | 31 | Infertility, recurrent | Severe PMS | Hypersensitivity to | Abolishment of PMS |
| | | habitual abortions | syndrome | estrone, estradiol | and atopic dermatitis |
| | | | | | symptoms. Prozac |
| | | | | | treatment was |
| | | | | | stopped. Weight loss |
| | | | | | of 7 kg. |
| | | | | | Spontaneous |
| | | | | | pregnancy with |
| | | | | | normal delivery. |
| 4 | 40 | Infertility, recurrent | Severe PMS | Hypersensitivity to | Mild PMS (VAS-4). |
| | | habitual abortions | syndrome | estrone, estriol, | Successful pregnancy |
| | | | (VAS 10) | estradiol and | and normal delivery. |
| | | | | progesterone | |
| 5 | 48 | Premenstrual | | Hypersensitivity to | Mild symptoms; |
| | | syndrome, hot | | estriol, estrone, and | imsomnia and fatigue |
| | | flashes (severe), | | progesterone | were absent. |
| | | night sweats | | | |
| | | (severe), insomnia, | | | |
| | | fatigue syndrome. | | | |
| 6 | 50 | Menopausal | | Hypersensitivity to | Mild hot flashes; |
| | | syndrome, | | estriol, estrone and | Insomnia and fatigue |
| | | characterized by hot | | estradiol. | are absent. |
| | | flashes (severe), | | | |
| | | insomnia, bloating, | | | |
| | | and fatigue | | | |
| 7 | 53 | Menopause, hot | | Hypersensitivity to | lessening of hot |
| | | flashes (severe), | | estrone, estradiol | flashes and heart |
| | | chronic urinary tract | | and progesterone | palpitations, normal |
| | | infection (UTI), | | | sleep had been |
| | | suspected interstitial | | | restored. |
| | | cystitis. | | | |

### Example 6

### Transdermal Diagnostic and Therapeutic System

In order to transdermally deliver the hormones described herein for the diagnosis of hypersensitivity to a female reproductive hormone and/or the treatment of medical condition associated with the hypersensitivity to a female reproductive hormone, the present inventors have devised a novel system which uses non-invasive epicutaneous, intra-epidermal or intradermal delivery of sex hormones.

The system is designed to deliver the female reproductive hormones to the epidermis or dermis layers of the skin while avoiding a systemic administration to the blood stream.

The uniqueness of the system is that it mimics injection into the skin by delivery of maximal drug dose in a shortest time.

Following is a non-limiting example of a system for diagnosing and treating according to the teachings of the invention.

For the preparation of a patch reservoir the dose range per spot should be 0.1 micrograms (μg)-200 μg for diagnosis; and 0.1 μg-1600 μg for treatment (by desensitization).

The dose range for hormonal administration in matrix per spot should be 0.1 μg-200 μg per 1 cm² area for diagnosis: and 0.8 μg-1600 μg per 1 cm²-30 cm² area for treatment.

Preparation of hormone for administration via patch reservoir, gel or spray (for treatment)—The female reproductive hormone is dissolved in a volume of 0.1 mL-1.5 mL of solvent which may contain an enhancer.

Preparation of hormone for administration via patch matrix (for treatment)—The female reproductive hormone is dissolved in a solid matrix containing enhancer and adhesives.

Time of hormone administration (for treatment and diagnosis)—From about 1 hour to about 48 hours.

Site of drug application—buttocks, abdomen, upper outer arm, or upper torso (excluding breasts), and/or thigh.

Diagnosis of sensitivity to hormones using skin test—Four hormones and solvent control in a reservoir patch (1 cm²) are applied onto the patient's thigh. The hormones are administered at luteal phase of the menstrual cycle. The drugs are dissolved in the 1.5 mL enhancer enabling the drug permeation into the epicutaneous layers in 1 hour. After one hour the patch is displaced and the skin reaction and symptom appearance are followed up daily for one month.

Desensitization treatment—Based on the results of the skin test patches containing appropriate hormones are applied at the same area of the skin test. Patches have the same structure and contain the same concentration of the drug. The size of the patch is increasing to contain a doubled dose of hormone. The patch is applied in a luteal phase, adhered for one hour and the skin reaction and symptom appearance are followed up daily for one month.

Next two treatments involve patches of growing sizes for doubling hormone dose.

Table 10 provides an example of doses used in diagnosing and treating the condition associated with hypersensitivity to a female reproductive hormone according to some embodiments of the invention.

TABLE 10

| | Patch size (assuming | |
|---|---|---|
| | the height is the same) | Hormone 1 |
| Skin test (diagnosis) | 1 cm² | 10 μg |
| 1^{st} treatment | 2 cm² | 20 μg |
| 2^{nd} treatment | 4 cm² | 40 μg |
| 3^{rd} treatment | 8 cm² | 80 μg |

The patch or the system may further include materials which reduce annoying irritation symptoms (like itching). For example, magnesium (Mg) at a dose of between about 10% to about 80% may be added (for further details see Hypertext Transfer Protocol://World Wide Web (dot) magnesiumforlife (dot) com/dosage (dot) shtml; and Hypertext Transfer Protocol://World Wide Web (dot) integratedhealth (dot) com/hpdspec/magnesium-oil (dot) html; each of which is hereby incorporated by reference in its entirety].

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Battist A. P. and Baldwin J. L., 2004, Clin. Mol. Allergy, 2:10;
2. Beaumont V, et al., 1992, Atherosclerosis, 94:147-152;
3. Davis D. L., et al., 1997, Environ Health Perspect. 105 Suppl. 3:571-6;
4. Govorukhina E M, 1986, Pruritus vulvae. Med. Sestra, 45:31-4;
5. Govorukhina E M, 1987, Pathogenesis and treatment of the premenstrual syndrome. Akush Ginekol (Mosk), 9:33-37;
6. Itsekson A., et al. 2004, J. Reprod. Med. 49: 195-199;
7. Lindsay S. 2006, Arch. Dermatol. 142:371-376;
8. Mabray C. R., et al., 1982, Obstet. Gynecol. 59:560-564;
9. Miller J. B., 1974, The journal of the Medical Association of the state of Alabama 44:57-60;
10. Roby et al., 2006, Am. J. Reprod. Immunol. 55: 307-13;
11. Schmidt et al., 1998, N. Engl. J. Med. 338:209-16;
12. Schoenmakers A., et al., 1992, Contact Dermatitis, 26:159-62;
13. Stranahan D., et al., 2006, Dermatitis, Vol. 17:32-42;
14. Wilkinson S. M., and Beck M. H., 1994, Dermatitis, 30:302-303;
15. Shelley W B., et al., 1995, J. of the American Academy of Dermatology, 32, 25-31.
16. Itsekson A., et al. 2007, Am. J. of Reproductive Immunology, 57:160-165.

1. A method of diagnosing a medical condition associated with hypersensitivity to a female reproductive hormone in a subject, with the proviso that the medical condition is not estrogen dermatitis, comprising:
(a) administering into a skin of the subject at least one female reproductive hormone, wherein said at least one female reproductive hormone comprises estriol or estrone, and;
(b) monitoring a reaction to said female reproductive hormone on said skin, wherein a presence of said reaction above a predetermined threshold is indicative of the presence of the medical condition in the subject.

2. A kit for diagnosing a medical condition associated with hypersensitivity to a female reproductive hormone in a subject, the kit comprising at least one female reproductive hormone, wherein said at least one female reproductive hormone comprises estriol or estrone, and a lesion indicator for assessing a reaction to said female reproductive hormone on a skin.

3. The method of claim 1, wherein said at least one female reproductive hormone further comprises estradiol and/or progesterone.

4. (canceled)

5. The method of claim 1 wherein said predetermined threshold comprises an increase in diameter of at least 5 mm in said reaction as compared to a control test.

6. The method of claim 1, wherein the medical condition comprises a premenstrual syndrome.

7. The method of claim 1, wherein the medical condition is selected from the group consisting of premenstrual dysphoric disorder (PMDD), pruritus vulvae, menstrual asthma, menstrual migraine, infertility, hot flashes, mastalgia, acne, premenopausal syndrome, menopausal syndrome, recurrent abortions, fatigue syndrome, unexplained preterm delivery, unexplained irritable bowel syndrome, endometriosis, idiopathic premature delivery, sympathovagal dysbalance in pregnancy (hypotension and/or hypertension), menstrual cycle related alterations in metabolic diseases (e.g. Diabetes), hyper-emesis gravidarum, idiopathic gingivitis, unexplained infertility, unexplained recurrent in vitro fertilization (IVF) failure, unexplained recurrent intrauterine insemination (IUI) failure, unexplained IVF side effects, unexplained IUI side effects, unexplained ovarian hyperstimulation, unexplained male infertility, hypomagnesaemia, menstrual related catamenial epilepsy, menstrual related pain condition, unexplained pain condition, fibromyalgia dysmenorrhea, migraine, menstrual cycle associated hyperosmia, menstrual cycle associated accidents, menstrual cycle associated confusion, menstrual cycle associated weight gain, premature ovarian failure, and unexplained ovarian failure.

8. A method of treating a medical condition associated with hypersensitivity to a female reproductive hormone in a subject, comprising:
(a) diagnosing the medical condition according to the method of claim 1; and
(b) subjecting the subject to a desensitization treatment to the hypersensitivity to the female reproductive hormone.

9. A computing platform for designing a treatment regimen against hypersensitivity to a female reproductive hormone, comprising:
(a) a data storage device storing data comprising skin test results of a subject against the female reproductive hormone
(b) a processing unit for designing treatment regimen against the hypersensitivity to the female reproductive hormone using said data stored on said data storage devise;
(c) a user interface being for displaying the treatment regimen.

10-13. (canceled)

14. The method of claim 8, wherein said desensitization treatment comprises three consecutive administrations of the female reproductive hormone into the skin of the subject.

15. The method of claim 14, wherein said three consecutive administrations include a first administration at a dosage of 0.1-55 microgram (μg) of the female reproductive hormone, a second administration at an identical or larger dosage of said first administration and a third administration at an identical or larger dosage of said second administration.

16. The method of claim 14, wherein the subject has a menstrual cycle with regular ovulation, and whereas an interval between said three consecutive administrations comprises a complete menstrual cycle.

17. The method of claim 14, wherein the subject is at menopause, and whereas an interval between said three consecutive administrations comprises one week.

18. The method of claim 14, wherein an interval between said three consecutive administrations comprises one week.

19. The method of claim 18, wherein the subject has a menstrual cycle with regular ovulation and whereas the first administration of said three consecutive administrations is effected during a luteal phase of said menstrual cycle, the second administration of said three consecutive administrations is effected following one week of said first administration, and the third administration of said three consecutive injections is effected following one week of said second administration.

20. The method of claim 18, wherein the subject is a non-fertile female.

21. The method of claim 20, wherein the subject has an irregular menstrual cycle or an unovulation.

22. The method of claim 18, wherein the subject has a non-menstrual cycle related medical condition.

23. The method of claim 14, wherein the subject has a menstrual cycle with regular ovulation, and whereas the first administration of said three consecutive administrations is effected during a luteal phase of said menstrual cycle, the second administration of said three consecutive administrations is effected following a complete menstrual cycle of said first administration, and the third administration of said three consecutive administrations is administered to the subject following a complete menstrual cycle of said second administration.

24-26. (canceled)

27. The method of claim 14, wherein said administration is effected by an intradermal injection and/or by a patch reservoir.

28-32. (canceled)

33. The method of claim 5, wherein said control test comprises administering into said skin a diluent of said female reproductive hormone.

34-37. (canceled)
